(19) United States
(12) Patent Application Publication
(10) Pub. No.: US 2018/0051068 A1
(43) Pub. Date: Feb. 22, 2018

(54) CalA BINDING AGENTS, CalA PEPTIDES AND METHODS OF USE

(71) Applicant: LOS ANGELES BIOMEDICAL RESEARCH INSTITUTE AT HARBOR-UCLA MEDICAL CENTER, Torrance, CA (US)

(72) Inventors: Scott Filler, Torrance, CA (US); Hong Liu, Torrance, CA (US); Norma Solis, Torrance, CA (US); Ashraf Ibrahim, Torrance, CA (US)

(21) Appl. No.: 15/543,967

(22) PCT Filed: Jan. 14, 2016
(86) PCT No.: PCT/US2016/013483

§ 371 Date: Jul. 14, 2017

Related U.S. Application Data
(60) Provisional application No. 62/104,021, filed on Jan. 15, 2015.

(51) Int. Cl. C07K 16/14; A61K 39/395; A61K 45/06; A61K 38/16

(52) U.S. Cl. CPC C07K 16/14; A61K 39/39575; A61K 45/06; A61K 2039/505; C07K 2317/34; C07K 2317/76; A61K 38/16

(57) ABSTRACT

Embodiments disclosed herein relate to compositions comprising extracellular thaumatin domain proteins, or portions thereof (e.g., CalA polypeptides, or portions thereof), and compositions comprising binding agents (e.g., antibodies, fragments thereof, and the like) that bind specifically to CalA polypeptides, and methods of using the same for preventing or treating a fungal infection.

### RELATED PATENT APPLICATIONS

This patent application claims the benefit of U.S. Provisional Patent Application No. 62/104,021 filed on Jan. 15, 2015, entitled CalA BINDING AGENTS, CalA PEPTIDES AND METHODS OF USE, naming Scott Filler, Hong Liu, Norma Solis, and Ashraf Ibrahim as inventors, and designated by Attorney Docket No. 022098-0436213. The entire content of the foregoing application is incorporated herein by reference, including all text, tables and drawings.

### GOVERNMENT SUPPORT

This invention was made with government support under institute contract/grant number 12525-07 awarded by NIAD. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 14, 2016, is named LaBioMedPCT0444709_ST25.txt and is 30.9 KB (31,722 bytes) in size.

### FIELD OF THE INVENTION

Embodiments of the invention relate to extracellular thaumatin domain proteins (CalA polypeptides, or portions thereof), or compositions comprising binding agents (e.g., antibodies, fragments thereof, and the like) that bind specifically to CalA polypeptides, and methods of using the same for preventing or treating a fungal infection.

### INTRODUCTION

Aspergillus fumigatus is an opportunistic pathogen that causes the majority of cases of invasive aspergillosis (IA) (Maschmeyer et al., 2007; Patterson et al., 2000). The incidence of this infection has risen significantly with the increasing number of immunosuppressed patients (Kousha et al., 2011; Marr et al., 2002). Despite current antifungal therapy, the mortality rate for IA remains unacceptably high (Maschmeyer et al., 2007; Patterson et al., 2000; Patterson et al., 2005; Pegues et al., 2001). Thus, there remains a great need for new, more effective antifungal agents for the prevention and treatment of fungal infections. IA is initiated by inhalation of conidia, which are small enough to be deposited in the alveoli. In the absence of an effective host immune response, these inhaled conidia germinate to form filamentous hyphae that then invade alveolar epithelial cells (Filler and Sheppard, 2006). Subsequently, these hyphae invade the blood vessels, causing thrombosis and tissue infarction, a characteristic feature of IA. The mechanisms by which A. fumigatus interacts with host cells are incompletely understood. Identifying the fungal proteins and host receptors that mediate A. fumigatus invasion of pulmonary epithelial and endothelial cells is critical because this information provides potential opportunities for novel therapeutic strategies to block host cell invasion.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows Germination of the GFP labeled indicated strains on glass coverslips (FIG. 1A), and A549 cells (FIG. 1B). Results are the mean±SD of 3 experiments, each of which analyzed at least 100 cells per strain in triplicate. *p<0.01 compared to Af293 or the ΔCalA+CalA complemented strain. The results demonstrate that CalA is required for normal germination.

FIG. 2 shows scanning electron micrographs of the indicated strains pre-germinated 18 hours in white RPMI and then incubated 6 hours on A549 epithelial cells. From left to right, the strains shown are Af293, ΔCalA and ΔCalA+CalA. The micrographs indicate that deletion of CalA results in abnormal hyphal tips.

FIG. 3 shows a graphical representation of the relative amount of Af293 (black bars), ΔCalA (white bars) and ΔCalA+CalA (grey bars) fungal cells endocytosed by A549 epithelial cells (FIG. 3A) or HUVECs (FIG. 3B) after incubation for 2.5 hours, after which the number of endocytosed organisms was determined by a differential fluorescence assay. The graphical representation shows the relative amount of endocytosed fungal cells as percent of the control (i.e. Af293 strain). The Result are the mean±SD of 3 experiments, each performed in triplicate. *p<0.05 compared to Af293 or the ΔCalA+CalA complemented strain. The results indicate that CalA is necessary for maximal A. fumigatus invasion of pulmonary epithelial cells and HUVECs.

FIG. 4 shows expression of pCalA-mCherry (left panels) on the cell surface of A. fumigatus during the interaction with A549 epithelial cells (top panels) and human umbilical vein endothelial cells (HUVEC)(bottom panels). DIC indicates visualization by Differential Interference Contrast (DIC) imaging.

FIG. 5A shows confocal microscopic images of CalA-mCherry heterologously expressed on the cell surface of a S. cerevisiae strain that co-expresses the ALS1 adhesin gene after overnight culture in SC medium with 2% galactose and 1% raffinose. FIGS. 5 B and C shows a graphical representation of fungal adherence (FIG. 5B) and fungal invasion (FIG. 5C) to HUVECs expressing ALS1 (white histogram bars) and ALS plus CalA (filled histogram bars). Results are the mean±SD of four experiments, each performed in triplicate. *P<0.01 vs. control. The results indicate that CalA mediates endothelial cell adherence and invasion.

FIG. 6A shows an immunoblot of biotin-labeled A549 cell surface proteins associated with hyphae of A. fumigatus Af293, ΔcalA and ΔcalA+calA strains. FIG. 6B, top panel, shows an immunoblot of A549 epithelial cell proteins eluted from the indicated strains. Blot was probed with antibody against β1 integrin.

FIG. 6B shows an immunoblot of endothelial cell proteins (bottom panel) and epithelial proteins (top panel) eluted from the Af293 (wild type), ΔCalA and ΔCalA+CalA strains of A. fumigatus as indicated at the top of the blots. The blot was probed with antibody against β1 integrin. The double band seen in the total membrane preparation (Total membrane) is representative of the mature processed form of β1 integrin (top band) and the β1 integrin precursor (bottom band). FIG. 6C shows a densitometric analysis of 3 to 5 replicate immunoblots similar to those shown in FIG. 6B. *P<0.04 vs. Af293 and the ΔcalA+calA complemented strain (two-tailed Student's t-test assuming unequal variances).

FIG. 6D shows an immunoblot of endothelial cell proteins (top panel) and epithelial cell proteins (bottom panel) eluted from the Af293 (wild type), ΔCalA and ΔCalA+CalA strains of A. fumigatus as indicated at the top of the blots. The blot was probed with an antibody against α5 integrin.

FIG. 6E shows a densitometric analysis of 3 to 5 replicate immunoblots similar to those shown in FIG. 6D. *P<0.04 vs. Af293 and the ΔcalA+calA complemented strain (two-tailed Student's t-test assuming unequal variances).

FIG. 6F shows confocal microscopic images of accumulation of β1 integrin and α5 integrin on A549 epithelial cells (Top panels) and HUVEC endothelial cells (bottom panel) during A. fumigatus infection. The results indicate that the integrin α5β1 complex interacts with A. fumigatus CalA and is required for maximal endocytosis of A. fumigatus.

FIG. 7A shows a histogram plot demonstrating endocytosis of S. cerevisiae expressing ALS1 (white bars) or S. cerevisiae expressing ALS1 and CalA (filled bars) by a β1 integrin knock-out mouse fibroblast cell line GD25 (GD25), and a β1 integrin expressing cell line β1AGD25 (β1AGD25). GD25 cells endocytosed very few S. cerevisiae cells that expressed Als1, with or without CalA, whereas β1AGD25 cells avidly endocytosed S. cerevisiae cells that expressed both Als1 and CalA.

FIG. 7B shows a histogram plot demonstrating endocytosis of an A. fumigatus wild type strain, Af293 (black histogram bars), an A. fumigatus mutant, ΔCalA (white histogram bars) and an A. fumigatus mutant complemented strain ΔCalA+CalA (grey histogram bars) by a β1 integrin knock-out mouse (integrin β1^{-/-}) fibroblast cell line GD25 (GD25), and a β1 integrin expressing cell line β1AGD25 (β1AGD25). Integrin β1-deficient GD25 cells poorly endocytosed wild-type A. fumigatus, the ΔcalA mutant, and the ΔcalA+calA complemented strains. Results are the mean±SD of three experiments, each performed in triplicate. *P<0.05 vs. ALS1 expressing S. cerevisiae or Af293 controls. The results of FIGS. 6C and 6D indicate that β1 integrin is a host receptor of CalA that mediates endocytosis and knocking out β1 integrin expression reduces this interactions.

FIG. 8A-C shows inhibition of endocytosis of A. fumigatus into A549 pulmonary epithelial cells (FIG. 8A), primary vascular endothelial cells (FIG. 8B) and a primary human pulmonary alveolar epithelial cell line, HPAEpiC (FIG. 8C) by an antibody that binds specifically to β1 integrin (grey histogram bars) or α5 integrin (white histogram bars) and the absence of inhibition by a control IgG antibody (black histogram bars). Results are the mean±SD of three experiments, each performed in triplicate. *P<0.001 vs. control IgG (two-tailed Student's t-test assuming unequal variances).

FIG. 8D-E shows the effects of siRNA negative control treatment (black bars) or siRNA knockdown of β1 integrin (grey bars) and α5 integrin (white bars) on the endocytosis of A. fumigatus by A549 pulmonary epithelial cell (FIG. 8D) and primary vascular endothelial cell (FIG. 8E). Results are the mean±SD of three experiments, each performed in triplicate. *P<0.005 vs. control siRNA (two-tailed Student's t-test assuming unequal variances).

FIG. 9, Germination of the indicated A. fumigatus strains in vivo. FIG. 9A shows a survival curve of cortisone acetate treated mice infected with A. fumigatus wild type (solid thick black line), ΔCalA mutant (broken line) and the ΔCalA+CalA complemented strain (thin grey line). A total 16 mice were tested with each strain in two independent experiments. *P<0.05 compared to Af293 and ΔCalA+CalA complemented strains (Log-rank test). FIG. 9B (Pulmonary fungal burdon) shows the relative amount of fungal DNA (y-axis) in lung homogenates of mice after 5 days of infection with the indicated strains of A. fumigatus (wild type (Af293), ΔCalA mutant and the ΔCalA+CalA complemented strain as indicated on the x-axis). Results are median of 8 mice per strain. Horizontal bars indicate the median value. *P<0.02 compared to Af293 and ΔCalA+CalA complemented strains (Wilcoxon rank sum test). The results indicate that CalA is required for normal virulence in a mouse model of invasive aspergillosis.

FIG. 9C shows the capacity of the ΔcalA mutant to germinate in vivo. Mice were infected intratracheally with 10⁷ conidia of the wild-type, ΔcalA mutant, and ΔcalA+calA complemented strains. After 12 h, the mice were sacrificed and thin sections of the lungs were stained with Gomori-methenamine silver to visualize the organisms and enable the extent of fungal germination to be quantified. It was determined that all three strains germinated similarly (FIG. 9C), indicating that CalA is dispensable for germination in vivo. Results are the mean±SD of 3 mice infected by each strain, analyzing at least 100 cells per mouse. FIGS. 9D-9F show Quantitative analysis of invasion into total lung tissue (FIG. 9D), alveoli (FIG. 9E), and bronchi (FIG. 9F) by the indicated A. fumigatus strains 12 h after intratracheal inoculation. Results are from a total of 10 lung sections from 3 mice infected with Af293, 11 sections from 3 mice infected with the ΔcalA mutant, and 8 sections from 2 mice infected with the ΔcalA+calA complemented strain. Horizontal lines indicate the median. *P<0.04 compared to Af293 and the ΔcalA+calA complemented strain (Wilcoxon rank sum test).

FIG. 10 A-B shows the effects of control antibody treatment (control IgG, black histogram bars) or anti-CalA₂ antibody treatment (white histogram bars) of epithelial cells (FIG. 10A) and endothelial cells (FIG. 10B) on CalA mediated endocytosis (y-axis) of A. fumigatus. Results are the mean±SD of three experiments, each performed in triplicate. FIG. 10C shows a survival curve of cortisone acetate treated mice treated with a control IgG antibody (solid line) and a polyclonal anti-CalA₂ antibody (broken line) infected with A. fumigatus (wild type) in an invasive aspergillosis model. *P<0.05 vs. Control. The results suggest that anti-CalA₂ antibody blocks A. fumigatus host cell invasion and protects mice from A. fumigatus infection.

FIG. 11 shows the relative levels of adherence (y-axis) of A. fumigatus (Af293) and ΔCalA to A549 cells (FIG. 11A) and laminin coated 6-well plates (FIG. 11B). Experiments were done in triplicates and repeated three times independently. The results indicate comparable levels of adherence of the two strains to A549 cells or a laminin coated plastic surface.

FIG. 12 shows staining of wild type A. fumigatus (Af293, Darkest line), ΔCalA (medium grey line) and ΔCalA+CalA strains (as indicated) with Alexa Fluor 488 labeled laminin as visualized by flow cytometry (FACs). The results indicate that knocking out CalA does not change the capability of swollen conidia (FIG. 12A) or germ tubes (FIG. 12B) binding to laminin. Strains of A. fumigatus were incubated with Alexa Fluor 488 labeled laminin, washed and fixed prior to visualization.

FIG. 13 shows endocytosis of the A. fumigatus strains Af293 (black bar), ΔCalA (white bar), ΔCalA+CalA (horizontal hashed bar), ΔCalB (diagonal hashed bar), ΔCalB (white spotted bar), ΔΔCalAB (solid grey shaded bar), and ΔΔCalAC (black spot filled bar, far right).

FIG. 14 shows an immunoblot of whole cell lysates of epithelial cells (FIG. 14A) and endothelial cells (FIG. 14B) treated with control siRNA (left), β1 integrin siRNA (middle) and α5 integrin siRNA as indicated below the figures. The immunoblots were visualized by staining with antibodies specific for Actin (bottom panel), β1 integrin (top panel) and α5 integrin (middle panel) as indicated to the right of the figures.

FIG. 15 shows a photomicrographs of Gomori-methenamine silver (GMS) stained sections of mouse lungs 12 hours after intratracheal inoculation with 10⁷ condia of A. fumigatus strains Af293 (left), ΔCalA (middle), and ΔCalA+CalA (right) as indicated at the top of each panel. Large insets show magnified images of the regions outlined by the smaller square boxes.

FIG. 16 shows the polypeptide amino acid sequence of CalA of Aspergillus fumigatus, strain Af293. The amino acid sequence represented by the polypeptides of Antigen 1 (Ag1) and Antigen 2 (Ag2) are underlined. Scale bar equals 10 μm.

FIG. 17 shows a multiple sequence alignment of CalA proteins obtained from Neosartorya fischeri (N. fischeri), Penicillium digitatum (P. digitatum) and nine species of Aspergillus. Identical amino acids are indicated by an asterisk, a : (colon) indicates conservation between amino acids having strongly similar properties (e.g., scoring >0.5 in the Gonnet PAM 250 matrix) and a (period) indicates conservation between groups of amino acids having weakly similar properties (e.g., scoring=<0.5 in the Gonnet PAM 250 matrix). The broken underline indicates the amino acid region represented by Antigen 1 (Ag1). The bold underline indicates the amino acid region represented by Antigen 2 (Ag2) which is enclosed in a box.

FIG. 18 shows confocal microscopic images of wild-type A. fumigatus Af293 expressing CalA-RFP after a 4 hr. incubation with A549 epithelial cells (top) or laminin coated coverslips (bottom), demonstrating that CalA is expressed on the cell surface of swollen conidia of A. fumigatus. DIC indicates visualization by Differential Interference Contrast (DIC) imaging.

FIG. 19a-e shows that the ΔcalA mutant has wild-type adherence. (FIG. 19, a-d) show adherence of swollen conidia (FIGS. 19, a and b) and germlings (FIGS. 19, c and d) of the indicted A. fumigatus strains to 6-well tissue culture plates containing A549 epithelial cells (FIGS. 19, a and c) or coated with laminin (FIGS. 19, b and d). Data are mean±SD of 3 experiments each performed in triplicate. (FIG. 19, e) shows flow cytometry analysis of adherence of swollen conidia to fluid-phase laminin. The indicated trains of A. fumigatus were incubated with AlexaFluor 488-labeled laminin, washed and then fixed. The fluorescent intensity was quantified by flow cytometry.

FIG. 20 demonstrates that deletion of calA has no effect on production of galactoseaminogalactan, hexosamine or hexose production. FIG. 20(a) shows amounts of galactosaminogalactan (y-axis) released into the medium from Af293 wild-type and calA mutants. FIG. 20(b) shows the percentage of hexosamine (y-axis) released into the medium. FIG. 20(c) shows the percentage of hexose (y-axis) released into the medium from Af293 wild-type and calA mutants. Results are mean±SD of 3 independent experiments.

FIG. 21 shows effects of different substrates on the morphology of A. fumigatus hyphae. Af293 (wild type), ΔCalA and ΔCalA+CalA strains of A. fumigatus are as indicated above each column of panels. FIG. 21 shows scanning electron micrographs of the indicated strains of A. fumigatus that had been germinated for 18 hours in RPMI 1640 medium and then incubated for 6 hours on either cover slips coated poly-D-lysine (positively charged, top row of panels) or uncoated glass coverslips (negatively charged, bottom row of panels) as indicated.

FIG. 22 shows susceptibility of Af293 (wild type), ΔCalA and ΔCalA+CalA strains of A. fumigatus (indicated to the left of each row of panels) to different cell wall and cell membrane stressors. Images of serial 10-fold dilutions of the indicated strains that were plated on to Sabouraud agar (Sab), 200 μg/ml Congo red, 300 μg/ml calcofluorwhite (CFW), 40 μg/ml Caspofungin, 0.01% SDS, 3 mM H₂O₂, 2 mg/ml protamine sulfate (protamine) and incubated at 37° C. for 2 days.

FIG. 23 a-b shows the ΔcalA mutant has wild-type susceptibility to killing by macrophage-like (FIG. 23a) and neutrophil-like HL-60 cells (FIG. 23b). Germlings of the indicated strains were incubated with HL-60 cells that had been differentiated into macrophage-like (FIG. 23a) or neutrophil-like (FIG. 23b) cells. After 2.5 hours, the percentage of surviving organisms was determined by quantitative culture. Percent fungal killing is shown on the Y-axis. The results are the mean±SD of 3 experiments, each performed in triplicate.

FIG. 24 shows integrin α5β1 binds to clinical isolates of A. fumigatus and is required for maximal host cell invasion. FIG. 24(a) shows immunoblots of A549 epithelial cell membrane proteins that had been eluted from the indicated clinical isolates of A. fumigatus. Blots were probed with an antibody against integrin β1 (top) and integrin α5 (bottom). FIG. 24(b) shows effects of an anti-integrin β1 antibody on the endocytosis of the indicated clinical isolates of A. fumigatus by A549 epithelial cells. Results are mean±SD of 3 experiments, each performed in triplicate. *P<0.01 compared to cells incubated with the control IgG (two-tailed Student's t-test assuming unequal variances).

### SUMMARY OF THE INVENTION

In some embodiments, provided herein is a pharmaceutical composition comprising a CalA binding agent and a pharmaceutical acceptable excipient, diluent, additive or carrier, where the binding agent specifically binds to a CalA polypeptide. In some embodiments, provided herein is a pharmaceutical composition comprising a CalA polypeptide and a pharmaceutical acceptable excipient, diluent, additive or carrier. In some embodiments, provided herein is a method of administering to a subject in need thereof, a pharmaceutical composition comprising a binding agent in an amount sufficient to prevent or treat a fungal infection, wherein the binding agent specifically binds to a CalA polypeptide. In some embodiments, provided herein is a method of administering to a subject in need thereof, a pharmaceutical composition comprising a CalA polypeptide in an amount sufficient to prevent or treat a fungal infection.

In certain aspects, the binding agent can be an antibody, or a binding fragment thereof. The antibody can be a polyclonal antibody, or binding fragment thereof or a monoclonal antibody, or binding fragment thereof. In certain aspects, the monoclonal antibody can be an IgG1, IgA or IgM isotype. In certain aspects, the monoclonal antibody can be a chimeric antibody, a rabbit antibody, a mouse antibody, a human antibody, or a humanized antibody. In some embodiments the binding agent comprises an aptamer, camelid, DARPin, or an affibody. In some embodiments the binding fragment comprises a Fab, Fab′, F(ab′)2, Fv or scFV fragment of an antibody.

In some embodiments of the pharmaceutical composition, the CalA polypeptide comprises the amino acid sequence of Antigen 2 (Ag2) and/or comprises a polypeptide having at least 76% identity, and in some embodiments at least 80%, 85% or 90% identity to the amino acid sequence of Antigen 2. In some embodiments, the CalA polypeptide comprises a polypeptide having the amino acid sequence represented by the formula, X₁QX₂X₃VLQFEYTX₄X₅X₆X₇TI, (SEQ ID NO.: 15) where X₁ is D, N, T, E or S; X₂ is D, S, A, T or Q; X₃ is D or N; X₄ is Q, E, or K; X₅ is S, D or A; X₆ is G or D; and X₇ is D, E or Q. In some aspects the CalA polypeptide comprises the amino acid sequence VLQFEYT (SEQ ID NO.: 16).

In some embodiments the pharmaceutical composition comprises one or more antifungal medications configured for administration to a mammal. In certain aspects the antifungal medication comprises a suitable polyene antimycotic, imidazole antifungal medication, triazole antifungal medication, allylamine antifungal medication, abafungin, an echinocandin antifungal medication, benzoic acid, a keratolytic agent, ciclopirox olamine, flucytosine, 5-fluorocytosine, griseofulvin, haloprogin, tolnaftate, undecylenic acid, crystal violet, tolnaftate, amphotericin B, anidulafungin, caspofungin, fluconazole, flucytosine, micafungin, posaconazole, voriconazole, derivatives thereof, analogues thereof and/or combinations thereof. In some embodiments the pharmaceutical composition comprises an adjuvant. In some embodiments a pharmaceutical composition as described herein is a vaccine.

### DETAILED DESCRIPTION

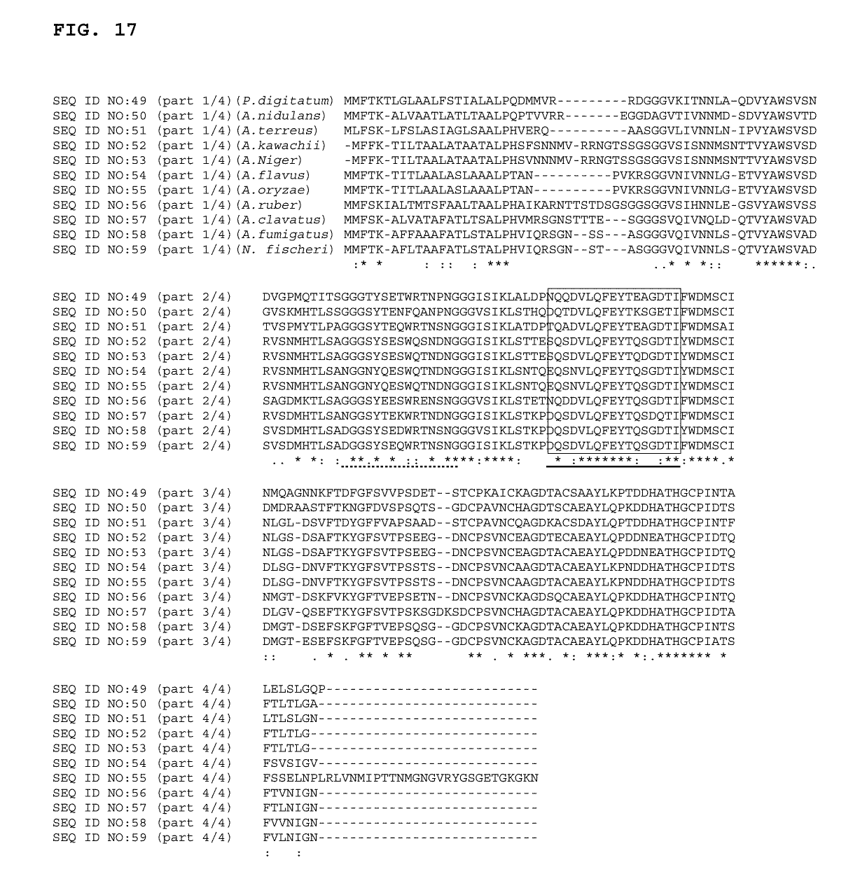

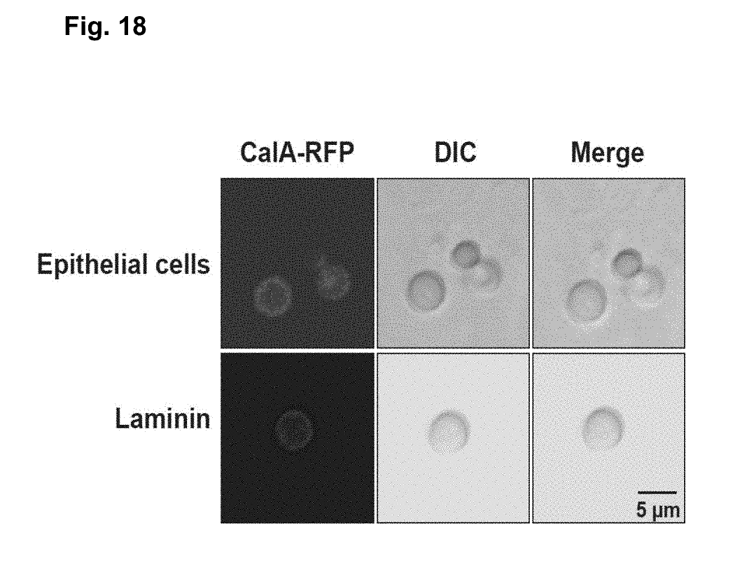

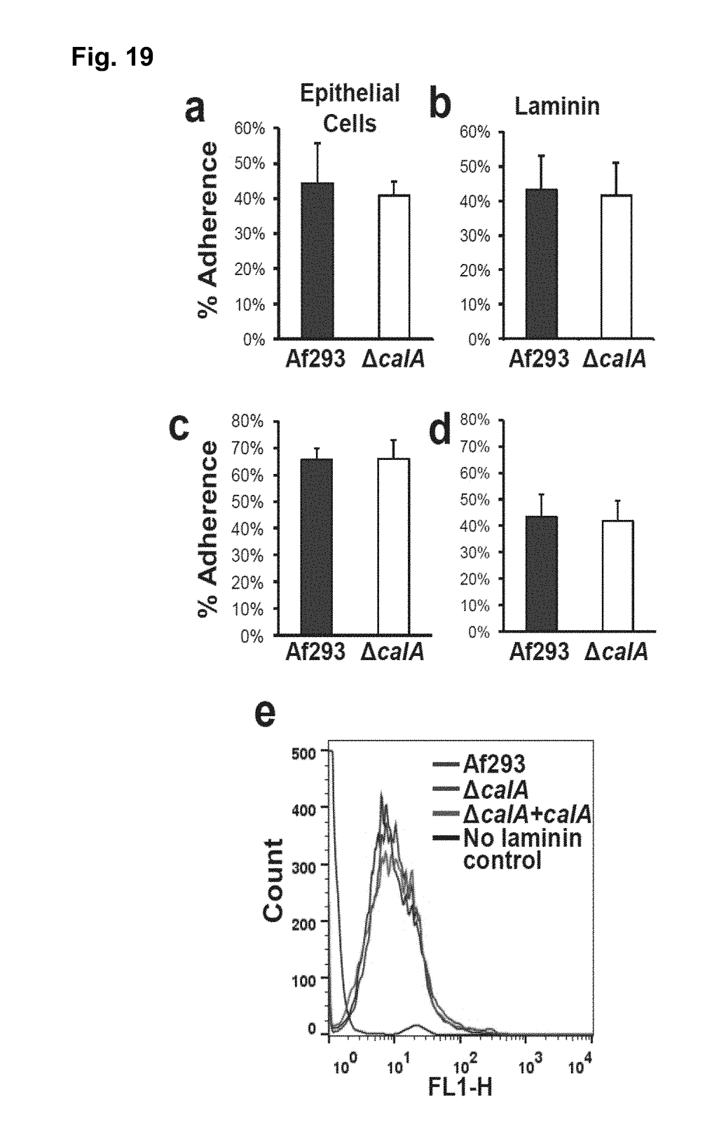

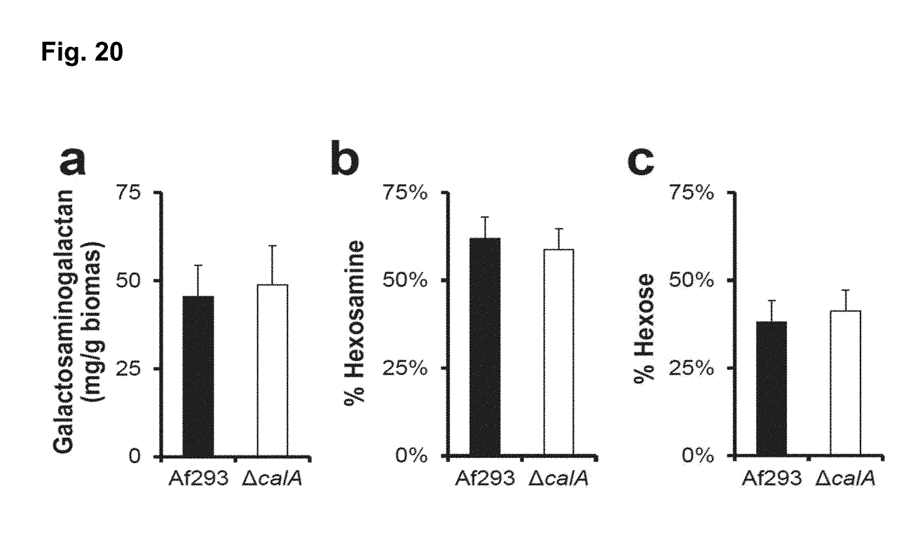

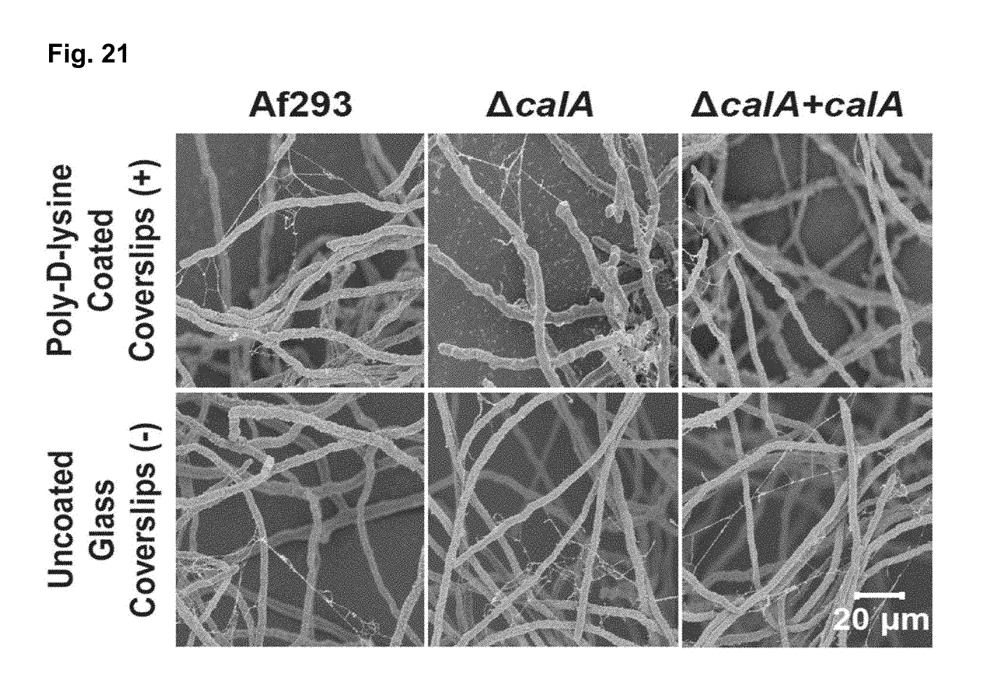

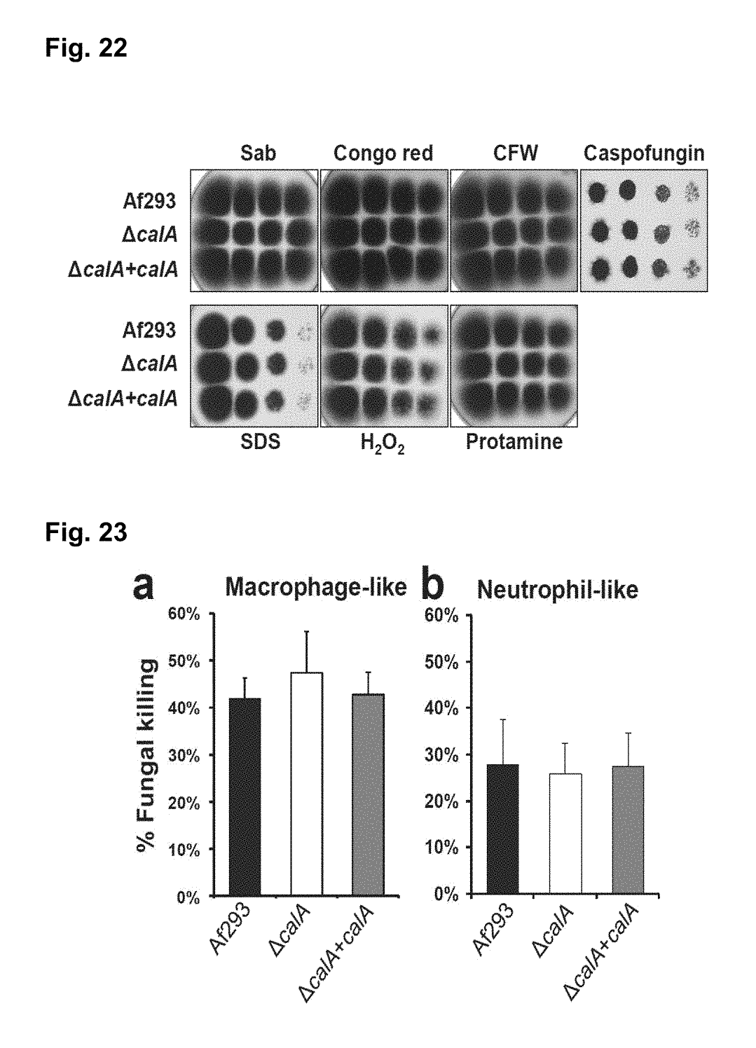

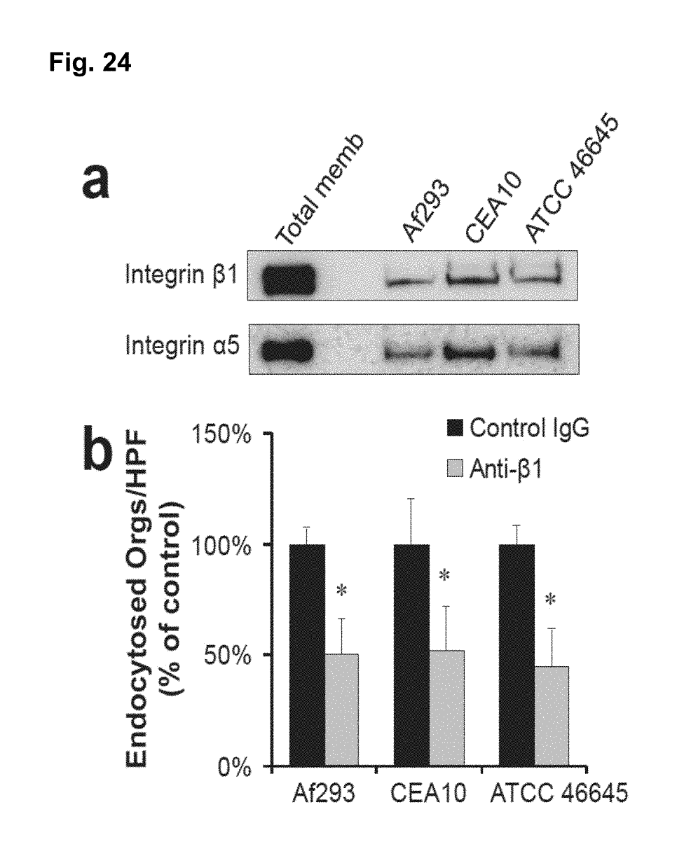

The extracellular thaumatin domain protein, sometimes referred to as CalA (i.e. CalA polypeptide) is expressed on the surface of pathogenic fungi as shown herein. CalA of Aspergillus fumigatus (FIG. 16) is a 177 amino acid protein that is highly conserved among Aspergillus and other fungal species. For example, CalA of A. fumigatus is 95% identical to CalA of Neosartorya fischeri, 78% identical to CalA of Aspergillus clavatus, and 73% identical to CalA of Aspergillus terreus. CalA of A. fumigatus also shares significant homology to CalA of Aspergillus ruber, Aspergillus flavus, Aspergillus niger, Aspergillus kawachii, Aspergillus oryzae, Aspergillus nidulans, Penicillium digitatum and several other species.

In Aspergillus nidulans, the CalA gene and its homolog CetA encode related fungal thaumatin-like proteins that are expressed in the cell wall of germinating cells. In A. nidulans, disruption of either CalA or CetA impairs fungal germination, and dual disruptions results in a synthetic lethal phenotype (Belaish et al., 2008). When CalA of A. fumigatus was heterologously expressed in Escherichia coli, the recombinant protein was found bind to laminin and murine lung cells (Upadhyay et al., 2009). However, prior to this publication, the function of CalA in mediating host cell interactions and virulence has not been examined.

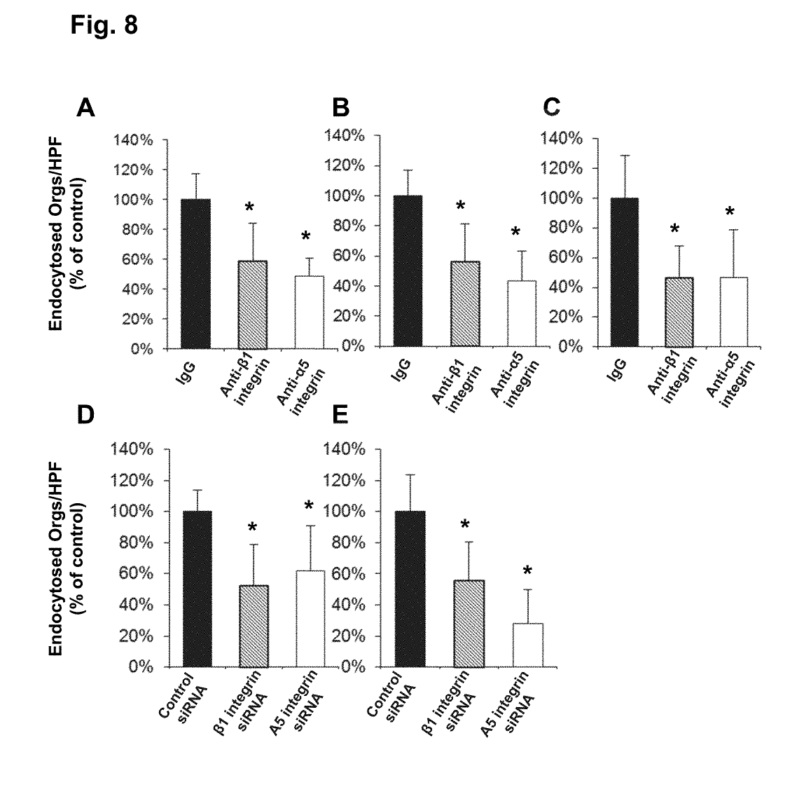

Studies performed herein (see Example 1) suggest that CalA is an adhesion that mediates invasion of, and endocytosis into mammalian host cells (e.g., epithelial and endothelial cells), in part by binding to integrins exposed on the cell surface of target mammalian host cells. CalA mediated endocytosis was shown to be inhibited by antibodies that specifically bind to cell surface integrins (e.g., see Example 1, FIG. 8). This data suggested that CalA mediated invasion of host cell might be blocked by antibodies directed to CalA.

A multiple sequence alignment revealed two regions of the CalA amino acid sequence that shared significant sequence homology among multiple species (see underlined regions, FIG. 17). Computer modeling algorithms predicted that these two regions were likely to contain surface exposed residues. Two peptides were synthesized that represented these regions. A first peptide having the amino acid sequence DGGSYSEDWRTNSNG (SEQ ID NO.: 48) and was termed Antigen 1 (Ag1) and a second polypeptide having the amino acid sequence DQSDVLQFEYTQSGDTI (SEQ ID NO.: 47) was termed Antigen 2 (Ag2). The 17 amino acid sequence of peptide Ag2 shared 100% identity with Cal A of N. fischeri, 94% identity with A. kawachii, 88% identity with A. clavatus, A. ruber, A. oryzae, A. flavus, A. niger, 82% identity with A. nidulans, 76% identity with A. terreus and P. digitatum. The internal epitope of Ag2 represented by the sequence VLQFEYT was 100% conserved among all species shown. For immunization purposes, both peptides included a C-terminal cysteine residue that was used to conjugate each peptide to keyhole limpet hemocyanin. The conjugated peptides were used to generate polyclonal antibodies in rabbits. The rabbit polyclonal antibodies were shown to specifically bind their corresponding unconjugated immunogens (i.e., Ag1 and Ag2) by ELISA (data not shown).

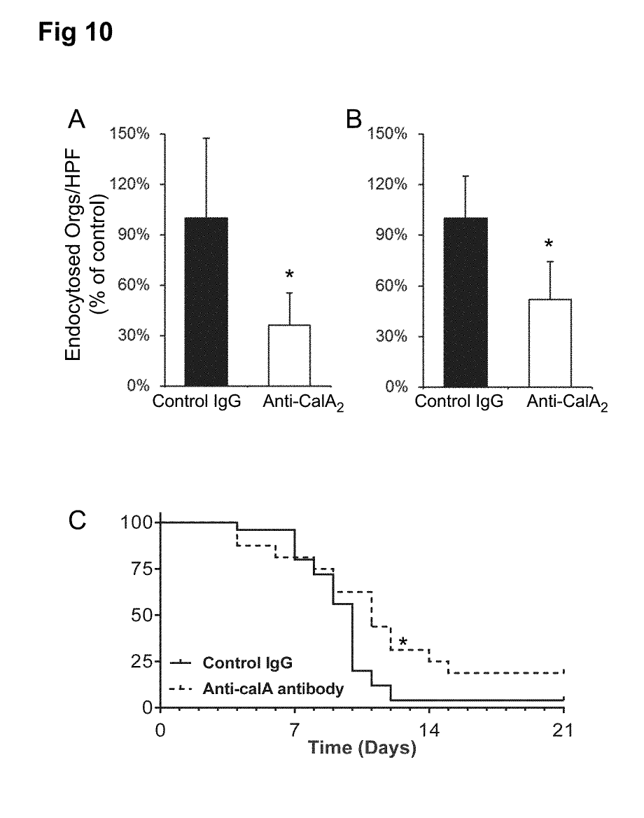

Rabbit polyclonal antibodies specific for Ag2 were shown to inhibit host cell invasion and endocytosis of Aspergillus (Example 1, FIGS. 10A & 10B). In addition, rabbit polyclonal antibodies specific for Ag2 were shown to delay infection of mice with Aspergillus and prolong survival of mice upon infection with Aspergillus (Example 1, FIG. 10C). Rabbit polyclonal antibodies specific for Ag1 failed inhibit host cell invasion, endocytosis and failed to delay infection or prolong survival of mice infected with Aspergillus.

The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

### Subjects

The term “subject” refers to animals, typically mammalian animals. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model, for example, animal models used for the study of fungals.

In some embodiments a subject or mammal is “at risk” of acquiring a fungal infection (e.g., a fungal infection). A mammal that is at risk may have increased risk factors for fungal infection, non-limiting examples of which include immunocompromised individuals or immune deficient subjects (e.g., bone marrow transplant recipients, irradiated individuals, subjects having certain types of cancers, particularly those of the bone marrow and blood cells (e.g., leukemia, lymphoma, multiple myeloma), subjects with certain types of chronic infections (e.g., HIV, e.g., AIDS), subjects treated with immunosuppressive agents, subjects suffering from malnutrition and aging, subjects taking certain medications (e.g. disease-modifying anti-rheumatic drugs, immunosuppressive drugs, glucocorticoids) and subjects undergoing chemotherapy), the like or combinations thereof).

In some embodiments a subject in need of a treatment or composition described herein is a subject at risk of a fungal infection and/or a subject that has a fungal infection. In some embodiments a subject in need of a treatment or composition described herein is infected with, or is suspected of being infected with, a fungus. In certain embodiments a binding agent or composition described herein is used to treat or prevent a fungal infection in a subject or a subject at risk of acquiring a fungal infection.

In some embodiments a subject in need of a treatment or composition described herein is a donor. In some embodiments a donor is healthy subject or a moderately healthy subject. In some embodiments a donor is free of a fungal infection. A donor may or may not be at risk of acquiring a fungal infection. In some embodiments a donor is an organ donor. In some embodiments a donor is preselected or predetermined to donate an organ, blood, bone marrow, serum, or the like to a subject who is at risk, or will become at risk of acquiring a fungal infection. Thus a donor is sometimes a subject in need of treatment or a composition described herein.

### Fungus

In some embodiments a fungus refers to any pathogenic fungus or a fungus capable of causing an infection in a subject. In some embodiments a fungus refers to any pathogenic fungus of the family Trichocomaceae, non-limiting examples of which include pathogenic fungi of the Genus Aspergillus, Byssochlamys, Capsulotheca, Chaetosartorya, Chaetotheca, Chromocleista, Cristaspora, Dendrosphaera, Dichlaena, Dichotomomyces, Edyuillia, Emericella, Erythrogymnotheca, Eupenicillium, Eurotium, Fennellia, Hamigera, Hemicarpenteles, Neocarpenteles, Neopetromyces, Neosartorya, Paecilomyces, Penicilliopsis, Penicillium, Petromyces, Phialosimplex, Rasamsonia, Sagenoma, Sagenomella, Sclerocleista, Sphaeromyces, Stilbodendron, Talaromyces, Thermoascus, Trichocoma, Warcupiella, mitosporic Trichocomaceae, the like and/or mixtures thereof. In some embodiments a fungus is pathogenic species of the genus Aspergillus, non-limiting examples of which include Aspergillus aculeatus, Aspergillus alliaceus, Aspergillus caesiellus, Aspergillus caespitosus, Aspergillus candidus, Aspergillus carneus, Aspergillus clavatus, Aspergillus deflectus, Aspergillus egyptiacus, Aspergillus fischerianus, Aspergillus flavus, Aspergillus foetidus, Aspergillus fumigatus, Aspergillus glaucus, Aspergillus ibericus, Aspergillus lentulus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowii, Aspergillus tamari, Aspergillus terreus, Aspergillus ustus, Aspergillus versicolor, the like and/or mixtures thereof. In some embodiments a fungus is a pathogenic species of the genus Candida non-limiting examples of which include Candida albicans, C. glabrata, C. rugosa, C. parapsilosis, C. tropicalis, C. dubliniensis, the like or mixtures thereof. In certain embodiments a fungus can be Cryptococcus neoformans, Histoplasma capsulatum, Pneumocystis jirovecii, Stachybotrys chartarum, Pseudallescheria boydii, Exophiala jeanselmei, Geotrichum candidum, Rhizopus oryzae, Mucor indicus, any pathogenic strain of the genus Mucor, Lichtheimia corymbifera, Syncephalastrum racemosum, Apophysomyces variabilis and the like.

Any suitable fungal infection can be treated by a method or composition herein include. Fungal infections can be systemic and/or local. Non-limiting examples local fungal infections include infections of the skin (epidermis, dermis, hypodermis, subcutaneous tissue), epithelial membranes, sinus membranes, ears, eyes, nose, throat, mouth, scalp, feet, nails, vagina, endometrium, urinary tract (e.g., bladder, urethra), the like or combinations thereof. Non-limiting examples systemic fungal infections include fungal infections of a tissue or organ (e.g., liver, kidney, heart, muscle, lung, stomach, large intestine, small intestine, testis, ovaries, brain), nervous tissue salivary glands, the like or combinations thereof.

### Binding Agents

A binding agent sometimes comprises a suitable antibody or an antibody fragment. As used herein the term ‘antibody’ is used to cover natural antibodies, polyclonal antibodies, monoclonal antibodies, recombinant antibodies, chimeric antibodies and CDR-grafted or humanized antibodies. In some embodiments, an antibody may be derived, obtained, isolated, or purified from any suitable species. Antibodies sometimes are IgG, IgM, IgA, IgE, an isotype thereof (e.g., lgG1, lgG2a, lgG2b or lgG3), or a mixture thereof. An antibody, whether natural or recombinant, can be polyclonal or monoclonal. In some embodiments an antibody, or fragment thereof is chimeric, humanized or bispecific. In some embodiments a binding agent comprises antibodies or portions thereof, chimeric antibodies, Fab, Fab′, F(ab′)2, Fv fragments, scFvs, diabodies, aptamers, synbodies, camelids, DARPins, affibodies, the like and/or combinations thereof. Chimeric antibodies often comprise a mixture of portions of binding agents or antibodies derived from different species. In some embodiments chimeric antibodies comprise fully synthetic portions or sequences of amino acids not found in native antibody molecules. In some embodiments chimeric antibodies comprise amino acid substitutions derived from antibodies of other species or, in some embodiments chimeric antibodies comprise amino acid substitutions added in an attempt to increase binding affinity (e.g., by an in vitro process of affinity maturation) or alter antibody function (e.g., to increase or decrease complement mediated or cell mediated cell lysis).

In some embodiments a binding agent comprises a chimeric antibody, humanized antibody, human antibody, or a portion or fragment thereof. Methods for generating chimeric and humanized antibodies also are known (see, e.g., U.S. Pat. No. 5,530,101 (Queen, et al.), U.S. Pat. No. 5,707,622 (Fung, et al.) and U.S. Pat. Nos. 5,994,524 and 6,245,894 (Matsushima, et al.)), which generally involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

An antibody fragment utilized as a binding agent sometimes is a Fab, Fab′, F(ab)′2, Dab, Fv or single-chain Fv (ScFv) fragment, and methods for generating antibody fragments are known (see, e.g., U.S. Pat. Nos. 6,099,842 and 5,990,296). In some embodiments, a binding agent comprises a single-chain antibody fragment, which can be constructed by joining a heavy chain variable region with a light chain variable region by a polypeptide linker (e.g., the linker is attached at the C-terminus or N-terminus of each chain) using recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. In certain embodiments the binding partner is an intact immunoglobulin, and in other embodiments the binding partner is a Fab monomer or a Fab dimer. For fragments or antibodies comprising a portion or all of a Fc region, a sensing region in a device may include one or more linkers linked to an amino acid or other portion of the Fc region.

The term “specifically binds” refers to a binding agent binding to a target peptide in preference to binding other molecules or other peptides in a particular assay in vitro assay (e.g., an Elisa, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

In some embodiments a carrier, antifungal medication, radio isotope and/or a polypeptide can be indirectly or directly associated with, or bound to (e.g., covalently bound to, conjugated to), a binding agent. Agents or molecules are sometimes conjugated to or bound to antibodies to alter or extend the in vivo half-life of an antibody or fragment thereof.

In some embodiments carriers or antifungal medications are bound to a binding agent by a linker. A linker can provide a mechanism for covalently attaching a carrier and/or an antifungal medications to a binding agent. Any suitable linker can be used in a composition or method described herein. Non-limiting examples of suitable linkers include: silanes, thiols, phosphonic acid, polyethylene glycol (PEG). Methods of attaching two or more molecules using a linker are well known in the art and are sometimes referred to as “crosslinking”. Non-limiting examples of crosslinking include an amine reacting with a N-Hydroxysuccinimide (NHS) ester, an imidoester, a pentafluorophenyl (PFP) ester, a hydroxymethyl phosphine, an oxiran or any other carbonyl compound; a carboxyl reacting with a carbodiimide; a sulfhydryl reacting with a maleimide, a haloacetyl, a pyridyldisulfide, and/or a vinyl sulfone; an aldehyde reacting with a hydrazine; any non-selective group reacting with diazirine and/or aryl azide; a hydroxyl reacting with isocyanate; a hydroxylamine reacting with a carbonyl compound; the like and combinations thereof.

### CalA Polypeptides

In some embodiments a CalA polypeptide comprises between about 15 and 250, between about 15 and 150, between about 15 and 100, between about 15 and 50, between about 15 and 30, between about 100 and 250, between 100 and 200, between about 120 and 200, or between about 150 and 200 amino acids in length.

In some embodiments a CalA polypeptide is a polypeptide amino acid sequence having at least 60% identity, at least 70% identity, at least 80% identity, at least 90% identity, at least 95% identity to the CalA polypeptide of FIG. 16. In some embodiments a CalA polypeptide consists of the CalA polypeptide amino acid sequence of FIG. 16. In some embodiments a CalA polypeptide comprises a polypeptide amino acid sequence having at least 70% identity, 76% identity, 82% identity, 88% identity, 90% identity, 95% identity, or 100% identity to amino acid sequence of Antigen 2 (Ag2). In some embodiments a CalA polypeptide comprises a polypeptide amino acid sequence having the amino acid sequence X₁QX₂X₃VLQFEYTX₄X₅X₆X₇TI (SEQ ID NO.: 15) where X₁ is D, N, T, E or S; X₂ is D, S, A, T or Q; X₃ is D or N; X₄ is Q, E, or K; X₅ is S, D or A; X₆ is G or D; and X₇ is D, E or Q. In some embodiments a CalA polypeptide comprises a polypeptide amino acid sequence having the amino acid sequence VLQFEYT (SEQ ID NO.: 16).

The term “percent identical” refers to sequence identity between two amino acid sequences. Identity can be determined by comparing a position in each two sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

In certain embodiments a CalA polypeptide is a CalA protein expressed by a wild type fungal strain or species of the genus Aspergillus, Penicillium or Neosartorya. In certain embodiments a CalA polypeptide is a CalA protein expressed by a wild type fungal species selected from one or more of A. fumigatus, A. kawachii, A. flavus, A. oryzae, A. ruber, A. terreus, A. niger, A. clavatus, and A. nidulans. In certain embodiments a CalA polypeptide is a CalA protein expressed by a wild type fungal species selected from Neosartorya fischeri or Penicillium digitatum.

### Antifungal Medications

Any suitable antifungal medication, or combinations thereof, can be used for composition or method described herein. In some embodiments an antifungal medication comprises a polyene antimycotic, non-limiting examples of which include amphotericin B, candicidin, filipin, hamycin, natamycin. nystatin, rimocidin, derivatives thereof, analogues thereof, the like or combinations thereof. In some embodiments an antifungal medication comprises an imidazole antifungal medication, non-limiting examples of which include bifonazole, butoconazole, clotrimazole, econazole, fenticonazole, isoconazole, ketoconazole, luliconazole, miconazole, omoconazole, oxiconazole, sertaconazole, sulconazole, tioconazole, derivatives thereof, analogues thereof, the like or combinations thereof. In some embodiments an antifungal medication comprises an triazole antifungal medication, non-limiting examples of which include albaconazole, efinaconazole, fluconazole, isavuconazole, itraconazole, posaconazole, ravuconazole, terconazole, voriconazole, derivatives thereof, analogues thereof, the like or combinations thereof. In some embodiments an antifungal medication comprises abafungin, derivatives thereof, analogues thereof, and the like. In some embodiments an antifungal medication comprises an allylamine antifungal medication, non-limiting examples of which include amorolfin, butenafine, naftifine, terbinafine, derivatives thereof, analogues thereof, the like or combinations thereof. In some embodiments an antifungal medication comprises an echinocandin antifungal medication, non-limiting examples of which include anidulafungin, caspofungin, micafungin, derivatives thereof, analogues thereof, the like or combinations thereof. In some embodiments an antifungal medication comprises benzoic acid, a keratolytic agent, ciclopirox olamine, flucytosine, 5-fluorocytosine, griseofulvin, haloprogin, tolnaftate, undecylenic acid, crystal violet, tolnaftate, derivatives thereof, analogues thereof, the like or combinations thereof. In some embodiments an antifungal medication comprises amphotericin B, anidulafungin, caspofungin, fluconazole, flucytosine, micafungin, posaconazole, voriconazole, derivatives thereof, analogues thereof, the like or combinations thereof.

### Pharmaceutical Compositions

In some embodiments a pharmaceutical composition comprises a binding agent that binds specifically to a CalA polypeptide and/or a CalA polypeptide as described herein. In some embodiments a pharmaceutical composition comprises a binding agent that binds specifically to a CalA polypeptide and an antifungal medication. In some embodiments a pharmaceutical composition comprises a CalA polypeptide and an antifungal medication.

In some embodiments a pharmaceutical composition comprises a suitable pharmaceutical acceptable excipient, diluent, additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agents include those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

In some embodiments a pharmaceutical composition comprises one or more suitable preservatives. In some embodiments a pharmaceutical composition is free of preservatives. In some embodiments a pharmaceutical composition, binding agent or CalA polypeptide is substantially free of serum proteins. In some embodiments a pharmaceutical composition, binding agent or CalA polypeptide is sterile. In some embodiments a pharmaceutical composition, binding agent or CalA polypeptide is lyophilized to a dry powder form, which is suitable for reconstitution with a suitable pharmaceutical solvent (e.g., water, saline, an isotonic buffer solution (e.g., PBS), and the like), which reconstituted form is suitable for parental administration (e.g., intravenous administration) to a mammal.

The pharmaceutical compositions described herein may be configured for administration in any suitable form and/or amount according to the therapy in which they are employed. For example, a pharmaceutical composition configured for parenteral administration (e.g. by injection or infusion), may take the form of a suspension, solution or emulsion in an oily or aqueous vehicle and it may contain formulator agents, excipients, additives and/or diluents such as aqueous or non-aqueous solvents, co-solvents, suspending solutions, preservatives, stabilizing agents and or dispersing agents. In some embodiments a pharmaceutical composition suitable for parental administration may contain, in addition to one or more active agents, excipients, non-limiting examples of which include starch—e.g. potato, maize or wheat starch or cellulose—or starch derivatives such as microcrystalline cellulose; silica; various sugars such as lactose; magnesium carbonate and/or calcium phosphate.

In some embodiments a pharmaceutical compositions described herein may be configured for topical, rectal, or vaginal administration and may include one or more of a binding and/or lubricating agent, polymeric glycols, gelatins, cocoa-butter or other suitable waxes or fats. In some embodiments, a pharmaceutical composition described herein is incorporated into a topical formulation containing a topical carrier that is generally suited to topical drug administration and comprising any suitable material known in the art. A topical carrier may be selected so as to provide the composition in the desired form, e.g., as a solution or suspension, an ointment, a lotion, a cream, a salve, an emulsion or microemulsion, a gel, an oil, a powder, or the like. It may be comprised of naturally occurring or synthetic materials, or both. A carrier for the active ingredient may also be in a spray form. It is preferable that the selected carrier not adversely affect the active agent or other components of the topical formulation. Non-limiting examples of suitable topical carriers for use herein can be soluble, semi-solid or solid and include water, alcohols and other nontoxic organic solvents, glycerin, mineral oil, silicone, petroleum jelly, lanolin, fatty acids, vegetable oils, parabens, waxes, and the like. Semisolid carriers preferably have a dynamic viscosity greater than that of water. Other suitable vehicles include ointment bases, conventional creams such as HEB cream; gels; as well as petroleum jelly and the like. If desired, and depending on the carrier, the compositions may be sterilized or mixed with auxiliary agents, e.g., preservatives, stabilizers, wetting agents, buffers, or salts for influencing osmotic pressure and the like. Formulations may be colorless, odorless ointments, lotions, creams, microemulsions and gels.

Ointments can be semisolid preparations which are typically based on petrolatum or other petroleum derivatives. The specific ointment base to be used, as will be appreciated by those skilled in the art, is one that will provide for optimum delivery of the active agent, and, preferably, will provide for other desired characteristics as well, e.g., emolliency or the like. As with other carriers or vehicles, an ointment base should be inert, stable, nonirritating and nonsensitizing. Ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (OAV) emulsions, and include, for example, cetyl alcohol, glyceryl monostearate, lanolin and stearic acid. Exemplary water-soluble ointment bases are prepared from polyethylene glycols (PEGs) of varying molecular weight, e.g., polyethylene glycol-1000 (PEG-1000). Oils of animal or plant origin such as peanut oil, mineral oil, soybean oil, or sesame oil, corn oil, or synthetic oils may be added.

Binding agents and/or peptides may be incorporated into lotions, which generally are preparations to be applied to the skin surface without friction, and are typically liquid or semiliquid preparations in which solid particles, including the active agent, are present in a water or alcohol base. Lotions can be suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. In some embodiments a lotion formulation for use in conjunction with the present method contains propylene glycol mixed with a hydrophilic petrolatum.

In some embodiments binding agents and/or CalA polypeptides are formulated as creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation can be a nonionic, anionic, cationic or amphoteric surfactant.

Binding agents and CalA polypeptides may be incorporated into microemulsions, which generally are thermodynamically stable, isotropic clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives and fatty alcohols. In some embodiments emulsifier/co-emulsifier combinations are selected from the group consisting of: glyceryl monostearate and polyoxyethylene stearate; polyethylene glycol and ethylene glycol palmitostearate; and caprilic and capric triglycerides and oleoyl macrogolglycerides. In certain embodiments a water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG, etc.

The term “an amount sufficient to prevent or treat a fungal infection” as used herein refers to the amount or quantity of active agents (e.g., a binding agent, CalA polypeptide, antifungal medication, and/or a combination of these active agents) present in a pharmaceutical composition that is determined high enough to prevent or treat a fungal infection and low enough to minimize unwanted adverse reactions. The exact amount of active agents or combination of active agents required will vary from subject to subject, depending on age, general condition of the subject, the severity of the condition being treated, and the particular combination of drugs administered. Thus, it is not always possible to specify an exact universal amount sufficient to prevent or treat a fungal infection for a diverse group of subjects. As is well known, the specific dosage for a given patient under specific conditions and for a specific disease will routinely vary, but determination of the optimum amount in each case can readily be accomplished by simple routine procedures. Thus, an appropriate “an amount sufficient to prevent or treat a fungal infection” in any individual case may be determined by one of ordinary skill in the art using routine experimentation.

### Vaccines and Adjuvants

In certain embodiments a composition comprising a CalA polypeptide induces an immune response directed to the CalA polypeptide when the CalA polypeptide, or a composition comprising the CalA polypeptide is administered to an immuno-competent subject. In some embodiments, an immune response is the production of one or more antibodies in an immuno-competent subject that bind specifically to a CalA polypeptide. In some embodiments, an immune response is the production of one or more pro-inflammatory cytokines in an immuno-competent subject that are produced in response to the presence of a CalA polypeptide. In some embodiments, an immune response is the production of one or more antigen reactive T-cells in an immuno-competent subject that are produced in response to the presence of a CalA polypeptide. The presence of an immune response to a CalA polypeptide can be measured by any suitable method known in the art. In certain embodiments a composition comprising a CalA polypeptide, portion or fragment thereof, is useful as a vaccine.

In certain embodiments, the compositions described herein comprise one or more adjuvants. In certain embodiments a composition comprising a CalA polypeptide comprises an adjuvant. Adjuvants are known in the art to further increase the immune response to an applied antigenic determinant (for a review on adjuvants, see, e.g., Montomoli, 2011, Expert Rev Vaccines 10: 1053-1061). Examples of suitable adjuvants include aluminum salts such as aluminum hydroxide and/or aluminum phosphate; oil-emulsion compositions (or oil-in-water compositions), including squalene-water emulsions, such as MF59 (see, e.g., WO 90/14837); saponin formulations, such as, for example, QS21 and Immunostimulating Complexes (ISCOMS) (see, e.g., U.S. Pat. No. 5,057,540; WO 90/03184, WO 96/11711, WO 2004/004762, WO 2005/002620); Toll-like receptor (TLR) agonists, e.g., a TLR7 agonist (see, e.g., WO 2012/117377, page 15-18, for examples), e.g., in combination with an aluminum salt, e.g., aluminum hydroxide to which the TLR agonist may be adsorbed; bacterial or microbial derivatives, examples of which are monophosphoryl lipid A (MPL), 3-0-deacylated MPL (3dMPL), CpG-motif containing oligonucleotides, ADP-ribosylating bacterial toxins or mutants thereof, such as E. coli heat labile enterotoxin LT, cholera toxin CT, Pertussis toxin, tetanus toxoid and the like.

In some embodiments, a pharmaceutical composition comprising a CalA polypeptide, or portion thereof, and an adjuvant is a vaccine. A vaccine comprising a CalA polypeptide and an adjuvant can be used to prevent or reduce the severity of a fungal infection in a subject. In some embodiments a subject in need thereof is treated with a sufficient amount of a vaccine comprising a CalA polypeptide, or portion thereof, and an adjuvant. In some embodiments a vaccine composition comprises an adjuvant. In certain embodiments, the adjuvant is an aluminum salt such as aluminum phosphate, aluminum hydroxide or a combination thereof. In some embodiments a CalA polypeptide is adsorbed onto an aluminum salt. Also, additional antigens may be adsorbed onto an aluminum salt. In some embodiments a CalA polypeptide is adsorbed onto aluminum hydroxide. In some embodiments a CalA polypeptide is adsorbed onto adsorbed onto aluminum phosphate. Typically, individual antigens are individually adsorbed onto an aluminum salt, and the components are thereafter mixed to form a vaccine formulation. This sometimes allows to prepare vaccines in which some antigens are adsorbed onto a first aluminum salt (e.g., Al(PO)4), while other antigens are adsorbed onto a second aluminum salt (e.g., Al(OH)3).

In some embodiments an adjuvant comprises one or more of dimethyl dioctadecyl-ammonium bromide (DDA); monophosphoryl lipid A (MPL); LTK63, lipophilic quaternary ammonium salt-DDA, DDA-MPL, aluminum salts, aluminum hydroxide, aluminum phosphate, potassium aluminum phosphate, Montanide ISA-51, ISA-720, microparticles, immunostimulatory complexes, liposomes, a detergent, a cytokine, a chemokine, virosomes, virus-like particles, CpG oligonucleotides, cholera toxin, heat-labile toxin from E. coli, lipoproteins, dendritic cells, IL-12, GM-CSF, nanoparticles illustratively including calcium phosphate nanoparticles, combination of soybean oil, emulsifying agents, and ethanol to form a nanoemulsion; AS04, ZADAXIN, or combinations thereof. In some embodiments an adjuvant comprises Freund's adjuvant, Stimulon® QS-21 (Aquila Biopharmaceuticals, Inc., Framingham, Mass.), MPL® (3-O-deacylated monophosphoryl lipid A; Corixa, Hamilton, Mont.), and/or interleukin-12 (Genetics Institute, Cambridge, Mass.). In some embodiments a CalA polypeptide is conjugated to an adjuvant.

In certain embodiments, a pharmaceutical composition, vaccine and/or CalA polypeptide comprises aluminum as an adjuvant, e.g., in the form of aluminum hydroxide, aluminum phosphate, aluminum potassium phosphate, or combinations thereof, in concentrations of 0.05-5 mg, or from 0.075-1.0 mg, of aluminum content per dose, for example.

A person of the ordinary skill in the art has a sufficient expertise to determine the dosage of a vaccines of the instant invention. A vaccine can be administered by any suitable route, non-limiting examples of which include subcutaneously, intramuscular, intra-venous, intra-dermal, intra-nasal or orally.

### Route of Administration and Formulation

The exact formulation and route of administration for a composition for use according to the methods of the invention described herein can be chosen by the individual physician in view of the patient's condition. See e.g., Fingl et al. 1975, in “The Pharmacological Basis of Therapeutics,” Ch. 1 p. 1; which is incorporated herein by reference in its entirety. Any suitable route of administration can be used for administration of a pharmaceutical composition or binding agent described herein. Non-limiting examples of routes of administration include topical or local (e.g., transdermally or cutaneously, (e.g., on the skin or epidermus), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranially, intraarticular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is often provided to a subject for self-administration or for administration to a subject by another (e.g., a non-medical professional). For example a composition described herein can be provided as an instruction written by a medical practitioner that authorizes a patient to be provided a composition or treatment described herein (e.g., a prescription). In another example, a composition can be provided to a subject wherein the subject self-administers a composition orally, intravenously or by way of an inhaler, for example.

Pharmaceutical composition or binding agents herein can be formulated to be compatible with a particular route of administration or use. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose). In certain embodiments, a composition herein is substantially free of a chelator (e.g., a zinc chelator, e.g., EDTA or EGTA).

Compositions for injection include sterile aqueous solutions (where water soluble) or dispersions and sterile powders (e.g., sterile lyophilized preparations) for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyethylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolonged absorption of injectable compositions. Polysorbate 20 and polysorbate 80 can be added into the formulation mixture, for example, up to 1%. Other non-limiting additives include histidine HCl, α,α-trehalose dehydrate.

Alternately, one can administer compositions for use according to the methods of the invention in a local rather than systemic manner, for example, via direct application to the skin, mucous membrane or region of interest for treating, including using a depot or sustained release formulation.

In some embodiments, a pharmaceutical composition comprising a binding agent can be administered alone. In other embodiments, a pharmaceutical composition comprising a binding agent can be administered in combination with one or more additional materials, for example, as two separate compositions or as a single composition where the additional material(s) is (are) mixed or formulated together with the pharmaceutical composition. For example, without being limited thereto, the pharmaceutical composition can be formulated with additional excipients, additional active ingredients, other pharmaceutical compositions, antifungal medications or other binding agents.

The pharmaceutical compositions can be manufactured by any suitable manner, including, e.g., by means of conventional mixing, dissolving, granulating, dragee-making, levigating, emulsifying, encapsulating, entrapping or tableting processes.

Pharmaceutical compositions for use in accordance with the invention thus can be formulated in any suitable manner using one or more physiologically acceptable carriers comprising excipients and auxiliaries, which facilitate processing of the active compounds into preparations which can be used pharmaceutically. Proper formulation can depend upon the route of administration chosen. In particular, any suitable formulation, ingredient, excipient, the like or combinations thereof as listed in “Remington's Pharmaceutical Sciences,” Mack Publishing Co., Easton, Pa., 18th edition, 1990. can be used with a composition described herein. The various binding agents and compositions described herein, alone or in combination, can be incorporated into or used with the materials described in Remington's. Any suitable techniques, carriers, and excipients can be used, including those understood in the art; e.g., in Remington's Pharmaceutical Sciences, above. The pages in the attached Appendix from Remington's Pharmaceutical Sciences are incorporated herein by reference in their entirety, including without limitation for all of the types of formulations, methods of making, etc.

### Formulations

In some embodiments, the composition may be formulated, for example, as a topical formulation. The topical formulation may include, for example, a formulation such as a gel formulation, a cream formulation, a lotion formulation, a paste formulation, an ointment formulation, an oil formulation, and a foam formulation. The composition further may include, for example, an absorption emollient.

In some embodiments, at least part of the affected area of the mammal is contacted with the composition on a daily basis, on an as-needed basis, or on a regular interval such as twice daily, three times daily, every other day, etc. The composition can be administered for a period of time ranging from a single as needed administration to administration for 1 day to multiple years, or any value there between, (e.g., 1-90 days, 1-60 days, 1-30 days, etc.). The dosages described herein can be daily dosages or the dosage of an individual administration, for example, even if multiple administrations occur (e.g., 2 sprays into a nostril).

Some embodiments relate to methods of treating or preventing fungal infection through administration of compositions described herein to the upper respiratory track/bronchi in a mammal in need thereof, for example, by contacting at least part of the upper respiratory tract/bronchi of a mammal with a therapeutically effective amount of a composition as described above or elsewhere herein. The composition can be, for example, formulated as an aerosol formulation, including formulated for use in a nebulizer or an inhaler. The composition further may include other pharmaceutically acceptable components such as a preservative.

Other embodiments relate to pharmaceutical preparation formulated as aerosol compositions that include, for example, a composition as described herein and an aerosolized pharmaceutically acceptable carrier solution or dry powder. The compositions may be formulated, for example, to be substantially absorbed by a bronchus. The compositions also may include, for example, one or more of dichlorodifluoromethane, trichlorofluoromethane, dichlorotetrafluoroethane, carbon dioxide, and the like. The compositions can be formulated for use in a nebulizer or an inhaler, for example.

In certain embodiments, the amount of a binding agent can be any sufficient amount to prevent, treat, reduce the severity of, delay the onset of or alleviate a symptom of a fungal infection as contemplated herein or a specific indication as described herein.

### Aerosolized Formulations

Compositions for use according to the methods of the invention can be, in some embodiments, aerosolized compositions. The aerosolized composition can be formulated such that the composition has increased solubility and/or diffusivity. The composition can comprise a carrier. A carrier can improve the absorption of the composition, change the viscosity of a composition, improve the solubility of the composition, or improve the diffusivity of a composition compared to a pharmaceutical composition that does not comprise a carrier.

Liquid pharmaceutically administrable compositions can, for example, be prepared by dissolving, dispersing, etc. a binding agent as defined above and optional pharmaceutical adjuvants in a carrier (e.g., water, saline, aqueous dextrose, glycerol, glycols, ethanol or the like) to form a solution or suspension. Solutions to be aerosolized can be prepared in any suitable form, for example, either as liquid solutions or suspensions, as emulsions, or in solid forms suitable for dissolution or suspension in liquid prior to aerosol production and inhalation.

For administration by inhalation, the compositions described herein can conveniently be delivered in the form of an aerosol (e.g., through liquid nebulization, dry powder dispersion or meter-dose administration The aerosol can be delivered from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

By non-limiting example water-based liquid formulations can include a binding agent alone or with non-encapsulating water soluble excipients. Simple formulations can also include organic-based liquid formulations for nebulization or meter-dose inhaler. By non-limiting example organic-based liquid formulations can include a binding agent, with or without non-encapsulating organic soluble excipients.

Simple formulations can also include dry powder formulations for administration with a dry powder inhaler. By way of non-limiting example, dry powder formulations can include a binding agent alone or with either water soluble or organic soluble non-encapsulating excipients with or without a blending agent such as lactose.

Formulations can include water-based liquid formulations for nebulization. Non-limiting examples of water-based liquid complex formulations can include a binding agent encapsulated or complexed with water-soluble excipients such as lipids, liposomes, cyclodextrins, microencapsulations, and emulsions.

Formulations can also include organic-based liquid formulations for nebulization or meter-dose inhaler. Non-limiting examples of organic-based liquid complex formulations can include a binding agent encapsulated or complexed with organic-soluble excipients such as lipids, microencapsulations, and reverse-phase water-based emulsions.

Formulations can also include low-solubility, water-based liquid formulations for nebulization. A non-limiting example low-solubility, water-based liquid complex formulations can include a binding agent as a low-water soluble, stable nano suspension alone or in co-crystal/co-precipitate excipient complexes, or mixtures with low solubility lipids, such as lipid nano suspensions.

Formulations can also include low-solubility, organic-based liquid formulations for nebulization or meter-dose inhaler. A non-limiting example low-solubility, organic-based liquid complex formulations can include A binding agent as a low-organic soluble, stable nano suspension alone or in co-crystal/co-precipitate excipient complexes, or mixtures with low solubility lipids, such as lipid nano suspensions.

Formulations can also include dry powder formulations for administration using a dry powder inhaler. A non-limiting example, complex dry powder formulations can include A binding agent in co-crystal/co-precipitate/spray dried complex or mixture with low-water soluble excipients/salts in dry powder form with or without a blending agent such as lactose.

Specific methods for simple and complex formulation preparation are described herein. Any suitable A binding agent, including those described herein, are preferably directly administered as an aerosol to the respiratory tract.

Any suitable device technology can be used to deliver, for example, a dry powder or a liquid aerosolized product comprising a binding agent or a pharmaceutical composition comprising a binding agent. Dry powder formulations in some circumstances can require less time for drug administration. Liquid formulations can have longer administration times.

For aqueous and other non-pressurized liquid systems, a variety of nebulizers (including small volume nebulizers) can be used to aerosolize the formulations. Compressor-driven nebulizers can utilize jet technology and can use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers generally rely on mechanical energy in the form of vibration of a piezoelectric crystal to generate respirable liquid droplets and are commercially available from, for example, Omron Healthcare, Inc. and DeVilbiss Health Care, Inc. Vibrating mesh nebulizers rely upon either piezoelectric or mechanical pulses to generate respirable liquid droplets. Commercial examples of nebulizers that could be used in certain embodiments include RESPIRGARD II®, AERONEB®, AERONEB® PRO, and AERONEB® GO produced by Aerogen; AERX® and AERX ESSENCE™ produced by Aradigm; PORTA-NEB®, FREEWAY FREEDOM™, Sidestream, Ventstream and I-neb produced by Respironics, Inc.; and PARI LC-PLUS®, PARI LC-STAR®, and e-Flow7m produced by PARI, GmbH. By further non-limiting example, U.S. Pat. No. 6,196,219, is hereby incorporated by reference in its entirety.

In some embodiments, the drug solution can be formed prior to use of the nebulizer by a patient. In other embodiments, the drug can be stored in the nebulizer in solid form. In this case, the solution can be mixed upon activation of the nebulizer, such as described in U.S. Pat. No. 6,427,682 and PCT Publication No. WO 03/035030, both of which are hereby incorporated by reference in their entirety. In these nebulizers, the drug, optionally combined with excipients to form a solid composition, can be stored in a separate compartment from a liquid solvent.

### Pharmaceutical Carriers

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism. In some embodiments, a pharmaceutical carrier for a composition described herein can be selected from castor oil, ethylene glycol, monobutyl ether, diethylene glycol monoethyl ether, corn oil, dimethyl sulfoxide, ethylene glycol, isopropanol, soybean oil, glycerin, zinc oxide, titanium dioxide, glycerin, butylene glycol, cetyl alcohol, and sodium hyaluronate.

Furthermore, the pharmaceutical compositions used herein can preferably be stable over an extended period of time, for example on the order of months or years. Compositions comprising a binding agent can, in some embodiments, comprise a preservative. The preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). The preservative can comprise an alkyl-mercury salt of thiosalicylic acid, such as thiomersal, phenylmercuric nitrate, phenylmercuric acetate or phenylmercuric borate. The preservative can comprise a parabens, such as methylparaben or propylparaben. The preservative can comprise an alcohol, such as chlorobutanol, benzyl alcohol or phenyl ethyl alcohol. The preservative can comprise a biguanide derivative, such as chlorohexidine or polyhexamethylene biguanide. The preservative can comprise sodium perborate, imidazolidinyl urea, and/or sorbic acid. The preservative can comprise stabilized oxychloro complexes, such as known and commercially available under the trade name PURITE®. The preservative can comprise polyglycol-polyamine condensation resins, such as known and commercially available under the trade name POLYQUART® from Henkel KGaA. The preservative can comprise stabilized hydrogen peroxide generated from a source of hydrogen peroxide for providing an effective trace amount of resultant hydrogen peroxide, such as sodium perborate tetrahydrate. The preservative can be benzalkonium chloride.

The preservative can enable a composition comprising a binding agent to be used on multiple occasions. The preservative can reduce the effects of one or more of acid exposure, base exposure, air exposure, heat, and light on a binding agent. The compounds and compositions used herein can include any suitable buffers, such as for example, sodium citrate buffer and/or sequestering agents, such as edetate disodium sequestering agent. Ingredients, such as meglumine, may be added to adjust the pH of a composition or binding agent described herein. Binding agents and compositions described herein may comprise sodium and/or iodine, such as organically bound iodine. Compositions and compounds used herein may be provided in a container in which the air is replaced by another substance, such as nitrogen.

### Dosages and Products

Certain embodiments provide pharmaceutical compositions suitable for use in the technology, which include compositions where the active ingredients are contained in an amount effective to achieve its intended purpose. A “therapeutically effective amount” means an amount to prevent, treat, reduce the severity of, delay the onset of or inhibit a symptom of a fungal infection. The symptom can be a symptom already occurring or expected to occur. Determination of a therapeutically effective amount is well within the capability of those skilled in the art, especially in light of the detailed disclosure provided herein.

In other embodiments, a therapeutically effective amount can describe the amount necessary for a significant quantity of the composition to contact the desired region or tissue where prevention or treatment of a fungal infection is desired.

The binding agents and compositions comprising binding agents as described herein can be administered at a suitable dose, e.g., at a suitable volume and concentration depending on the route of administration. Within certain embodiments of the invention, dosages of administered binding agents can be from 0.01 mg/kg (e.g., per kg body weight of a subject) to 500 mg/kg, 0.1 mg/kg to 500 mg/kg, 0.1 mg/kg to 400 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a binding agent can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. Volumes suitable for intravenous administration are well known.

In some embodiments a binding agent or a pharmaceutical composition comprising a binding agent that is formulated for topical or external delivery can include higher amounts of binding agent. For example pharmaceutical composition comprising a binding agent that is formulated for topical administration may comprise at least 0.1 mg/ml, at least 1 mg/ml, at least 10 mg/ml, at least 100 mg/ml or at least 500 mg/ml of a binding agent.

The compositions can, if desired, be presented in a pack or dispenser device, which can contain one or more unit dosage forms containing the active ingredient. The pack can for example comprise metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions comprising a compound of the invention formulated in a compatible pharmaceutical carrier can also be prepared, placed in an appropriate container, and labeled for treatment of an indicated condition.

### Kits

In some embodiments the binding agents, compositions, formulations, combination products and materials described herein can be included as part of kits, which kits can include one or more of pharmaceutical compositions, binding agents, and formulations of the same, combination drugs and products and other materials described herein. In some embodiments the products, compositions, kits, formulations, etc. can come in an amount, package, product format with enough medication to treat a patient for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

The invention provides kits including pharmaceutical compositions of the invention, combination compositions and pharmaceutical formulations thereof, packaged into suitable packaging material. A kit optionally includes a label or packaging insert including a description of the components or instructions for use in vitro, in vivo, or ex vivo, of the components therein. Exemplary instructions include instructions for a method, treatment protocol or therapeutic regimen.

A kit can contain a collection of such components, e.g., two or more conjugates alone, or in combination with another therapeutically useful composition (e.g., an anti-proliferative or immune-enhancing drug). The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics (PK) and pharmacodynamics (PD). Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Kits can additionally include other components. Each component of the kit can be enclosed within an individual container and all of the various containers can be within a single package. Invention kits can be designed for cold storage. Invention kits can further be designed to contain host cells expressing fusion polypeptides of the invention, or that contain nucleic acids encoding fusion polypeptides. The cells in the kit can be maintained under appropriate storage conditions until the cells are ready to be used. For example, a kit including one or more cells can contain appropriate cell storage medium so that the cells can be thawed and grown.

### EXAMPLES

### Example 1

A. fumigatus invades host cells by inducing its own endocytosis (Lopes Bezerra and Filler, 2004). In the current study, we found that CalA functions as an invasin and is required for maximal invasion of both epithelial and endothelial cells. In addition, we identified integrin α5β1 as one of the host receptors that interact with CalA and mediates host cell endocytosis. Furthermore, CalA is required for maximal for virulence in a mouse model of invasive pulmonary aspergillosis, and an anti-CalA antibody directed to peptide Ag2 blocks A. fumigatus invasion of host cells in vitro increases the survival of IA mice. Thus, CalA is an A. fumigatus invasin that plays central role in virulence.

Material and Methods

Strains, Medium and Growth Conditions.

A. fumigatus strain Af293 (a generous gift from P. Magee, University of Minnesota) was used as the wild-type strain in all experiments. All strains were grown on Sabouraud dextrose agar (Difco) at 37° C. for 7 d prior to use. Conidia were harvested by rinsing with phosphate-buffered saline (PBS) (Corning Cellgro) containing 0.1% Tween 80 (Sigma-Aldrich) and enumerated using a haemacytometer. To obtain germlings for the adherence or uptake assay, 1×107 freshly harvested conidia were inoculated into 20 ml Sabouraud Dextrose broth (Difco, Detroit, Mich.) in 150-mm petri dishes. The petri dishes were incubated at 37° C. for 4 h and then stored at 4° C. overnight. The next day, the petri dishes were incubated at 37° C. for an additional 1.5 h. Next, the germlings were rinsed, removed from the plate with a cell scraper, resuspended in PBS and counted for use.

Strain Construction.

A split marker strategy was used to disrupt the 534 bp CalA (Afu3g09690) protein coding sequence (Catlett et al., 2002). DNA fragments encompassing 1,502 bp upstream and 1,529 bp downstream of CalA were amplified from genomic DNA of A. fumigatus strain Af293 by high-fidelity PCR using primers CalA-P1, CalA-P2 and CalA-P3, CalA-P4. The sequences of all PCR primers used in the experiments are listed in Table 1. Each fragment was cloned into the entry vector pENTR™/D-TOPO (Invitrogen). Subsequently, to construct the 5′ and 3′portions of the disruption cassette (pCalA-HY and pCalA-YG, respectively), LR clonase reactions were performed according to the manufacturer's instructions to introduce the two fragments into the pHY-Pr-att and pYG-Tr-att vectors, respectively (Gravelat et al., 2012). Next, a DNA fragment containing the upstream Ca/A flanking region and the 5′ portion of the hygromycin resistance cassette was amplified by high-fidelity PCR from pCalA-HY using primers CalA-P1 and HY. Similar, a DNA fragment containing the downstream Ca/A flanking region and the 3′ portion of the hygromycin resistance cassette was amplified from pCalA-YG using primers CalA-P4 and YG. A. fumigatus was transformed with both fragments by protoplasting. Transformants were selected on osmotically stable medium (OSM) plates containing 8 μg/ml hygromycin. Hygromycin-resistant clones were screened for disruption of Ca/A by colony PCR using primers CalA-RT-F and CalA-RT-R.

TABLE 1
(SEQ ID NOs.: 17-44)

| Primer name | Primer sequence (5′ - 3′) |
|---|---|
| CalA-P1 | CACCAGTACGTCGGTCAATCTTG |
| CalA-P2 | ACTGAGGATGTAGAGACTG |
| CalA-P3 | CACCTCAACTCTTGCTTGATGC |
| CalA-P4 | ACTGCTTCTGCATCATCAG |
| CalB-P1 | CACCTGGAGTGGTACATGCG |
| CalB-P2 | AGTCATGACTCATACGGC |
| CalB-P3 | CACCAGTCTGATCTACAGAGAG |
| CalB-P4 | TGAGTTCTAGTCCATGGC |
| CalC-P1 | CACCACGTGAGTACTGATGAGTG |
| CalC-P2 | AGACTGTAGCACAATGGC |
| CalC-P3 | CACCGTGAGGTACAGCTCTGC |
| CalC-P4 | AGGCAGTCTCGAGCATGG |
| HY | GGATGCCTCCGCTCGAAGTA |
| YG | CGTTGCAAGACCTGCCTGAA |
| BL | TGATGAACAGGGTCACGTC |
| LE | AAGTTGACCAGTGCCGTTC |
| CalA-RT-F | TCACCAAGGCCTTCTTCG |
| CalA-RT-R | AGCCGTGGGTGGCATGGTC |
| CalB-RT-F | AACTGATAGGACTCAGCG |
| CalB-RT-R | TCCAGATCGTCAACAACAT |
| CalC-RT-F | TGAGACGTACACCGAGAC |
| CalC-RT-R | AGGTCACGGATGAGCAAC |
| CalA-Com-F | ATTGCGGCCGCTCCATTCGAGGCAAGGATC |
| CalA-Com-R | TAAGCGGCCGCAGACTCTGGACTGGAGAC |
| CalA-mCherry-F | TAAGAGCTCCCATTCGAGGCAAGGATC |
| CalA-mCherry-R | TATGATATCGTTGCCAATGTTCACCAC |
| sCalA-RFP-F | TAGACTAGTATGATGTTCACCAAGGCC |
| sCalA-RFP-R | ATTGAGCTCTTAGGCGCCGGTGGAGTG |

The same strategy was used to knock out calB (Afu8g01710) and calC (Afu3g00510). 1460 bp downstream and 1720 bp upstream DNA fragments of CalB open reading frame were amplified with paired primers CalB-P1, CalB-P2 and CalB-P3, CalB-P4. 1527 bp upstream and 1461 downstream DNA fragments of CalA open reading frame were amplified with CalA-P1, CalA-P2 and CalA-P3, CalA-P4. Similar to replacing CalA with hygromycin B phosphotransferase (hpt), to make the ΔcalB or ΔcalC strain, calB or calC was replaced with HPH gene in wild type Af293 background. To make ΔΔCalAB or ΔΔCalAC double strains, two gateway vectors pBL and pLE were used instead of pHY-Pr-att and pYG-Tr-att vectors (Gravelat et al.). And calB or calC was replaced with phleomycin resistance gene (ble) in ΔCalA background. Primers CalB-RT-F, CalB-RT-R and CalA-RT-F, CalA-RT-R were used to screen the transformed clones.

To complement the ΔCalA mutant, the intact CalA open reading frame, including 2004 bp of upstream and 613 bp of downstream sequence, was amplified from genomic DNA by high-fidelity PCR using primers CalA-Com-F and CalA-Com-R. After NotI digestion, the resulting fragment was cloned into plasmid p402, which contains the phleomycin resistance gene (Richie et al., 2007). The resulting plasmid was transformed into the ΔCalA mutant. Hygromycin- and phlemycin-resistant clones were selected and the integration of intact wild type allele CalA was detected by whole cell PCR using primers CalA-Com-F and CalA-Com-R.

To construct the pCalA-mCherry expression strain in A. fumigatus, a 2534 bp DNA fragment containing 2003 bp of the CalA promoter region and 531 bp open reading frame was amplified with primers CalA-mCherry-F and CalA-mCherry-R. Next, the fragment was cloned into Phleo-mCherry plasmid at the SacI-EcoRV sites. The resulting plasmid, pCalA-mCherry, was subsequently transformed into Af293, and the integration of the plasmid was confirmed by whole cell PCR using primers CalA-mcherry-F and CalA-mCherry-R.

To generate a strain of S. cerevisiae that expressed CalA-mCherry, the CalA-mCherry fusion DNA fragment was amplified from plasmid pCalA-mCherry by high-fidelity PCR with primers sCalA-mCherry-F and sCalA-mCherry-R. This fragment was cloned into pESC-LEU (Agilent techonologies) at the SpeI-SacI sites. The resulting plasmid was used to transform S. cerevisiae S150-2B and a strain of S. cerevisiae that expressed the C. albicans ALS1 adhesin gene (Fu et al., 1998). The construction of S. cerevisiae strain that expressed Candida albicans Als1 under the promoter of ADH1 was described previously (Sheppard et al., 2004). The control strains of S. cerevisiae were transformed by backbone vector pESC-LEU alone. All S. cerevisiae strains were cultured in SC medium at 30° C. Expression of CalA-RFP was induced by growth in SC medium containing 2% galactose and 1% raffinose for overnight.

To construct GFP-expressing strains of A. fumigatus for the invasive assays, a GFP expressing plasmid with ble phleomycin resistance (GFP-Phlo) was used to transform Af293 and the ΔCalA mutant. Similarly, a GFP expressing plasmid with pyrithiamine resistance (GFP-PTRI) was used to transform the ΔCalA+CalA complemented strain. To make the GFP-PTRI, the eGFP gene was cloned into pPTRI (Takara Bio Inc). To obtain the GFP expressing strains, the phelomycin or pyrithiamine-resistant clones were examined under epifluorescence.

Tissue Culture.

The A549 type II pneumocytes cell line was purchased from the American Type Culture Collection (ATCC) and grown in F-12 K medium (ATCC) containing 10% fetal bovine serum (Gemini Bio-Products), streptomycin and penicillin (Irvine Scientific). The endothelial cells were isolated from human umbilical cord veins and grown in M199 medium (Gibco) containing 10% fetal bovine serum (Gemini Bio-Products), 10% bovine calf serum (Gemini Bio-Products), and 2 mM L-glutamine with penicillin and streptomycin (Irvine Scientific) (Lopes Bezerra and Filler, 2004). The GD25 and β1AGD25 cell lines were gifts from Dr. Deane F. Mosher at the University of Wisconisn-Madison. The GD25 cell line was grown in DMEM (Corning), containing 10% fetal bovine serum, streptomycin and penicillin, and the β1AGD25 cell line was grown with the same medium plus 10 μg/ml puromycin (A.G. Scientific, Inc.). Primary human pulmonary alveolar epithelial cells (HPAEpiC) were purchased from ScienceCell Research Laboratories and cultured in Alveolar Epithelial Cell medium (ScienceCell Research Laboratories).

Germination Assay.

To investigate the germination rate of different strains, conidia from GFP labeled strains were suspended in F-12 K medium at 10⁵/ml. 1 ml of conidia suspension was added to glass coverslips or confluent A549 cells on glass coverslips in a 24-well plate and incubated at 37° C. in 5% CO₂. At various time points, the medium was aspirated and the cells were fixed in 3% paraformaldehyde in PBS. The wells were examined by epifluorescence microscopy, and the number of organisms proceeding a germ tube was determined at least 1 conidial diameter in length. At least 100 organisms were counted on each coverslip. Each experiment was performed in triplicate and repeated 3 times.

Scanning Electron Microscopy.

For electronic microscopy, 5×10⁵ conidia/ml of Af293, ΔCalA and the ΔCalA+CalA complemented strains were pre-germinated in RPMI 1640 medium at 37° C. for 18 h. Next, the hyphae were harvested and incubated for 6 h on A549 cells or glass coverslips. After rinsing the coverslips three times with PBS, they were fixed overnight with 2.5% glutaraldehyde in 0.1 M sodium cacodylate buffer at 4° C. Subsequently, the samples were dehydrated and critical-point dried. Finally, the dried coverslips were sputter coated with Au—Pd and imaged with a Hitachi S-3000 N scanning electron microscope.

Adherence Assay.

The capacity of different A. fumigatus and S. cerevisiae strains to adhere to A549 cells and laminin was determined as previously described method (Gravelat et al.). Briefly, swollen conidia were prepared by growing 5×10⁵ conidia/ml in F-12 K medium at 37° C. for 4 h. CalA-mCherry over-expression and control strains of S. cerevisiae were cultured overnight in SC medium containing 2% galactose and 1% raffinose. Next, 3×10² organisms in 1 ml of HBSS/Tween 20 0.01% (Irvine Scientific) was added to each well of a 6-well plate, which was either coated with laminin (Sigma-Aldrich) or contained confluent A549 epithelial cells. After incubation for 30 min at 37° C. in 5% CO₂, the wells were washed three times with HBSS in a standard manner to remove non-adherent fungi, and then overlaid with YPD agar. The number of adherent organisms was determined by colony counting. Adherence was determined as a percentage of the original inoculums, which was measured by quantitative culture. Each assay was performed in triplicate and repeated at least three separate occasions.

Uptake Assay.

The number of organisms that were endocytosed by A549 pulmonary epithelial cells, endothelial cells, GD25, β1GD25 cells and HPAEpiCs was determined using a minor modification of our standard differential fluorescence assay (Phan et al., 2007). Briefly, different host cells were cultured on fibronectin-coated glass coverslips and then infected with 10⁵ germlings of the various GFP expressing strains of A. fumigatus. After incubation for 2.5 hours, the cells were washed with 1 ml HBSS to remove non-adherent fungi, and fixed with 3% paraformaldehyde. The noninternalized organisms were stained with a polyclonal rabbit anti-A. fumigatus serum (Meridian Life Science, Inc.) followed by anti-rabbit antibody conjugated with Alexa Fluor 568 (Life Technologies) (Lopes Bezerra and Filler, 2004). The coverslips were mounted inverted on a microscope slide and observed under epifluorescence. The number of organisms endocytosed by the host cells was determined by substracting the number of noninternalized organism (which fluoresced red) from the total number of organisms (which fluoresced green). At least 100 organisms were counted on each coverslip. The experiments were performed in triplicate and repeated at least three times.

The uptake of S. cerevisiae strains by endothelial cells was determined as previously described (Phan et al., 2007). The cells were infected with 10⁵ S. cereviase cells for 1.5 h and then fixed with 3% paraformaldehyde in PBS. The noninternalized cells were stained with an anti-C. albicans rabbit serum (Biodesign International) that had been conjugated with Alexa 568 (Invitrogen). This antiserum also recognizes S. cerevisiae. Next, the endothelial cells were permeablized in 0.1% (vol/vol) Triton X-100 in PBS, after which both the internalized and the non internalized organisms were stained with the anti-C. albicans rabbit serum conjugated with Alexa 488 (Invitrogen). The coverslips were mounted inverted on a microscope slide, viewed under epifluorescence to determine the number of organism endocytosed by the endothelial cells as above.

Fluorescent Labeling of Laminin.

For this study, 1 mg laminin was dialyzed overnight at 4° C. under constant shaking and then labeled with Alexa Fluor 488 (Invitrogen) following the manufacture's protocol. The labeled protein was pooled and protein content was measured using the Bio-Rad protein assay reagent.

Flow Cytometry.

Binding of the different A. fumigatus strains to laminin was analyzed by flow cytometry as described previously (Bouchara et al., 1997). In this assay, 107 swollen conidia or germlings were incubated with 250 μl of 50 μg/ml Alexa Fluor 488 conjugated laminin or PBS control at 37° C. for 1 hour in a shaker. Afterward, the organisms were washed three times with PBS and fixed with 3% paraformaldehyde in PBS. The fluorescent intensity of the organisms was quantified by flow cytometry. Fluorescence data for 10,000 cells of each strain were collected.

Isolation and Identification of Epithelial and Endothelial Cell Membrane Proteins that Bind to A. fumigatus.

Epithelial cell and endothelial cell membrane proteins that bound to A. fumigatus were isolated and affinity-purified as previously described (Phan et al., 2005). Briefly, cell surface proteins of A549 cells and endothelial cells were labeled with Ez-Link Sulfo-NHS-LS Biotin (Pierce) and extracted by 5.8% octyl-glucopyranoside (w/v) (Sigma). Epithelial proteins that bound to A. fumigatus germ tubes were eluted with 6 M urea, separated by SDS/PAGE, and detected by immunoblotting with anti-biotin antibody. The prominent bands were excised and identified by NanoLC-MS/MS (UCLA Molecular Instrumentation Center). The capacity of wild type, ΔCalA and the ΔCalA+CalA complemented strains to bind to the putative host cell receptors was analyzed by immunoblotting the eluted proteins with antibodies against β1 integrin (ab52971; Abcam) and α5 integrin (AB1928; Millipore).

Inhibition of Host Cell Endocytosis by Antibody Blocking or siRNA Knockdown Assay.

To determine the role of integrin α5β1 in mediating host cell endocytosis, the epithelial cells, endothelial cells and HPAEpiC cells were incubated with 10 μg/mL of anti-β1 integrin (6S6, Millipore) or 25 μg/mL anti-α5 integrin blocking anbody (NM-SAM-1, Millipore) 45 min before infection. Control cells were incubated with mouse IgG at 25 μg/mL. To knock down β1 integrin or α5 integrin, A549 epithelial cells and endothelial cells were transfected with random control siRNA (Qiagen), β1 integrin siRNA (sc-35674; Santa Cruz Biotechnoloy) or α5 integrin siRNA (sc-29372; Santa Cruz Biotechnology) using Lipofectamine 2000 (Invitrogen). The efficiency of siRNA knockdown of the two proteins was verified by immunoblotting of whole cell lysates with the anti-β1 and anti-α5 antibodies.

Confocal Microscopy.

The expression of CalA on the cell surface of A. fumigatus was determined using the strain expressing CalA-mCherry. Germlings of this strain were incubated with A549 cells and endothelial cells for 2.5 h, after which the medium was aspirated and the organisms were fixed in 3% paraformaldehyde in PBS. After washing, the coverslip was mounted on a glass slide and imaged by confocal microscopy. The expression of CalA on cell surface of S. cerevisiae was observed by Als1 plus CalA expression strain. CalA-mCherry was induced to express under SC medium with 2% galactose and 1% raffinose overnight.

The accumulation of integrin β1, integrin α5 around the cell surface of A. fumigatus was visualized as previously described (Phan et al., 2007). Briefly, epithelial or endothelial cells were infected with 10⁵ A. fumigatus germlings. After 2.5 h, the cells were fixed by 4% of paraformaldehyde or methanol, blocked with 5% goat serum (vol/vol), and sequentially incubated with antibody against integrin β1 or integrin α5 (Integrin β1 antibody, catalog #ab52971, abcam; Integrin α5 antibody, catalog #CBL497, EMD Millipore), followed by the appropriate secondary antibodies that had been labeled with either Alexa Fluor 647 or Alexa Fluor 488. The cells were imaged by confocal microscopy. The organisms were visualized by DIC imaging.

Anti-CalA Antibody Inhibition of Host Cell Endocytosis.

The rabbit anti-CalA polyclonal antibody was produced commercially (Promab Biotechnologies, Inc.). Briefly, two rabbits per peptide were immunized subcutaneously with peptides DQSDVLQFEYTQSGDTI (Ag2) (SEQ ID NO.: 47) or DGGSYSEDWRTNSNG (Ag1) (SEQ ID NO.: 48) that had been conjugated to keyhole limpet hemocyanin via a C-terminal cysteine residue added solely for this purpose. For the first immunization (day 1), 1 mg of conjugated peptide was mixed with Freund's complete adjuvant and injected subcutaneously. On days 21, 35, 49, 63, 77 and 91 the rabbits were boosted by subcutaneous injection of 0.5 mg peptide in Freund's incomplete adjuvant. After the rabbits developed sufficient antibody titers against the peptides, they were terminally exsanguinated and the Ig in the serum was precipitated with ammonium saturated ammonium sulfate. Saturated ammonium sulfate was prepared by dissolving 238 grams of ammonium sulfate in 250 ml of boiling distilled water. This was allowed to cool to 4° C. and stored refrigerated until ready for use. Saturated ammonium sulfate was added drop wise to the rabbit serum at 4° C. until equal volumes of ammonium sulfate and serum were combined to make a 50% NH4SO4 precipitate. The mixture was stirred for 30-60 minutes at 2-6° C. The mixture was then centrifuged at 3000×g for 10-15 minutes in a refrigerated centrifuge. The pellet containing isolated rabbit polyclonal antibody was re-suspended in 1×PBS. The procedure resulted in an anti-Ag1 (Anti-CalA₁) rabbit polyclonal antibody that was raised to the Ag1 peptide antigen and which bound specifically to unconjugated Ag1, and a second anti-Ag2 (Anti-CalA₂) rabbit polyclonal antibody that was raised to the Ag2 peptide antigen and which bound specifically to unconjugated Ag2. The specificity and relative affinities of Anti-CalA₁ and Anti-CalA₂ binding to their respective unconjugated antigens were determined by ELISA (data not shown).

Murine Model of Invasive Aspergillosis.

The virulence of ΔCalA was compared with wild type Af293 and ΔCalA+CalA complemented strain in a mouse model of invasive aspergillosis. Male Balb/C mice (Taconic Laboratories), weighting 19-21 g were used. The mice were immunosuppressed by administering 5 mg of cortisone acetate (Sigma-Aldrich) subcutaneously every other day, starting on 4 days prior to infection for a total of 5 doses (Liu et al.). To prevent bacterial infections, enrofloxacin (Baytril, Western Medical Supply) was added to the drinking water of mice to a final concentration of 0.005% the day before immunosuppression was initiated. The mice were infected by placing them for 1 hour in an acrylic chamber into which 12 ml of 1×10⁹ conida ml⁻¹ were aerosolized. Controls mice were immunosuppressed, but were not infected. 11 mice were infected with each strain. Shortly after infection, 3 mice form each group were sacrificed, and their lungs were harvested, homogenized and quantitatively cultured to verify conidia delivery to the lung. The remaining mice were monitored for survival. The survival experiments were repeated twice and the results were combined.

To determine the pulmonary fungal burden, mice were immunosuppressed and 8 mice were infected with each strain of A. fumigatus as above. After 4 days of infection, the mice were sacrificed and their lung were harvested and homogenized in 750 μl lysis solution (ZR Fungal/Bacterial DNA MiniPrep™, Epigenetics, USA) by gentle MACS dissociator (Miltenyi Biotec, Germany). To obtain fungal DNA, the homogenates were processed follow the direction of the kit. qPCR was performed using a real-time PCR protocol with Aspergillus specific primers ASF1 5′-GCA CGT GAA ATT GTT GAA AGG-3′(SEQ ID NO.: 45) and ADR1 5′-CAG GCT GGC CGC ATT G-3′ (SEQ ID NO.: 46) targeting the 28S rRNA gene (Williamson et al., 2000) and SYBR Green dye (Applied Biosystems). Relative fungal DNA content was quantified by the 2^{-ΔΔCT} method using GAPDH as the reference.

Intratracheal infection model was used to determine the germination of wild type, ΔCalA mutant and ΔCalA+CalA complemented strains. The same steroids treatment regimen was used as the inhalation model. Mice were anesthetized by 10 mg/kg xylazine (LLOYD laboratories, IO) and ketamine (Clipper Distributing Company, LLC. MO). 107 conidia were given to mouse by intratracheal infection. 3 mice were infected by each strain. After 12 h infection, mice lungs were isolated, fixed and GMS stain was performed for photo microscopy.

The same mouse model of invasive aspergillosis was used to determine the protective function of Anti-CalA2 antibody. Before infection, 8 mice were injected intraperitoneally with 0.1 mg (0.1 mg/kg body weight), 0.3 mg (0.3 mg/kg body weight) and 10 mg (10 mg/kg body weight) of the Anti-CalA₂ antibody or control rabbit IgG. The survivals of the two groups was monitored for 21 days. All three doses delayed the onset of fungal infection and protected mice from fungus induced death. The results of mice injected with 0.1 mg of Anti-CalA₂ antibody are shown in FIG. 10C. The survival experiments were repeated three times and the results were combined. Of note, the Anti-CalA₁ antibody at concentrations of 0.1 mg/kg to 10 mg/kg body weight failed to inhibit fungal infection and failed to provide protection to mice in the mouse model of invasive aspergillosis.

The animal studies were approved by the Institutional Animal Use and Care Committee, and performed according to the National Institute of Health guidelines for animal housing and care.

Susceptibility to Calcofluor White, Nikkomycin and Caspofungin.

To test the susceptibility of ΔCalA strain to different stresses, serial 10-fold dilutions of conidia from different A. fumigatus strains ranging from 10⁵ to 10² cells in a volume of 5 μl were spotted onto Sabouraud agar plates supplemented with 300 μg/ml Calcofluor White (Sigma), 75 μg/ml nikkomycin Z (Sigma) or 40 μg/ml caspofungin (Bellavida Pharmacy). Fungal growth was analyzed after incubation at 37° C. for 2 days.

Statistical Analysis.

The data from the in vitro experiments were analyzed by the two-tailed t-test. The survival data were analyzed using the Log-Rank test. A P-value of ≦0.05 was considered to be significant.

Results

CalA is an Orthologue of CalA and CetA in A. nidulans, and Required for Normal Conidial Germination

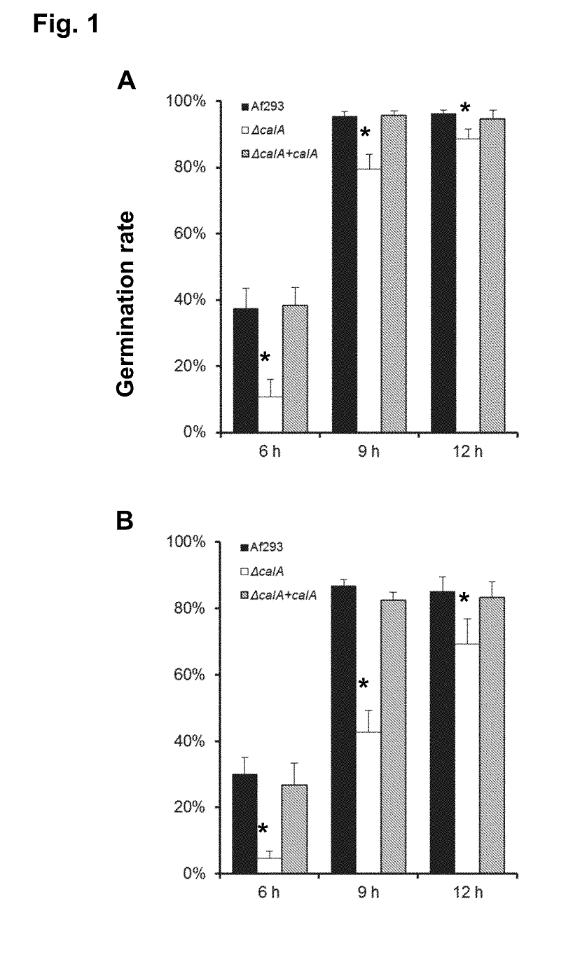

To investigate the function of CalA in A. fumigatus, we constructed a mutant strain in which the protein coding region of CalA was deleted. In addition, the radial growth and conidiation of the resulting mutant were similar to the wild-type strain. However, CalA and CetA are required for normal conidial germination in A. nidulans (Belaish et al., 2008), and the amino acid sequence of A. fumigatus CalA is 41.9% identical to AnCalA and 58.9% identical to AnCetA. Therefore, we investigated the capacity of the Af293 wild-type, ΔCalA mutant and ΔCalA+CalA complemented strains were observed by growing on glass coverslips or A549 epithelial. After 6 hour incubation, 37% spores of wild-type germinated, but only 11% spores of ΔCalA mutant germinated. And this germination defect of ΔCalA mutant can be restored by adding back of an intact copy of CalA. At long incubation time, the germination rate of the ΔCalA mutant eventually caught up with that of the wild type strain. As shown in FIG. 1A, after 9 h incubation, 80% conidia of the ΔCalA mutant were germinated as well as 95% of the conidia of controls and after 12 h incubation, the percentage germination of ΔCalA mutant was 89% and that of the control was 96%. In contrast to deletion both CalA and CetA led to profound germination defect in A. nidulans, at least 89% of the conidia of the ΔCalA mutant can germinate, even though the germination is delayed. This result indicates that CalA is involved in regulating A. fumigatus germination but different from its homologues in A. nidulans.

In addition, we examined the germination of the three strains when interacting with lung epithelial cells. We found that when wild type A. fumigatus conidia contacted with A549 epithelial cells, they germinated more slowly than on glass cover slips and only about 85% of conidia can germinate to form hyphae (FIG. 1B). Upon interaction with A549 cells, the ΔCalA mutant has a bigger germination defect when compared to the control strains. For example, at 9 h incubation time, only half cells of ΔCalA mutant germinated compared to wild type A. fumigatus. Collectively, these results indicate that CalA regulates A. fumigatus conidial germination, particularly during fungus and host cell interactions.

Deletion of CalA Results in Abnormal Hyphal Tip Formation when A. fumigatus Interacts with A549 Epithelial Cells

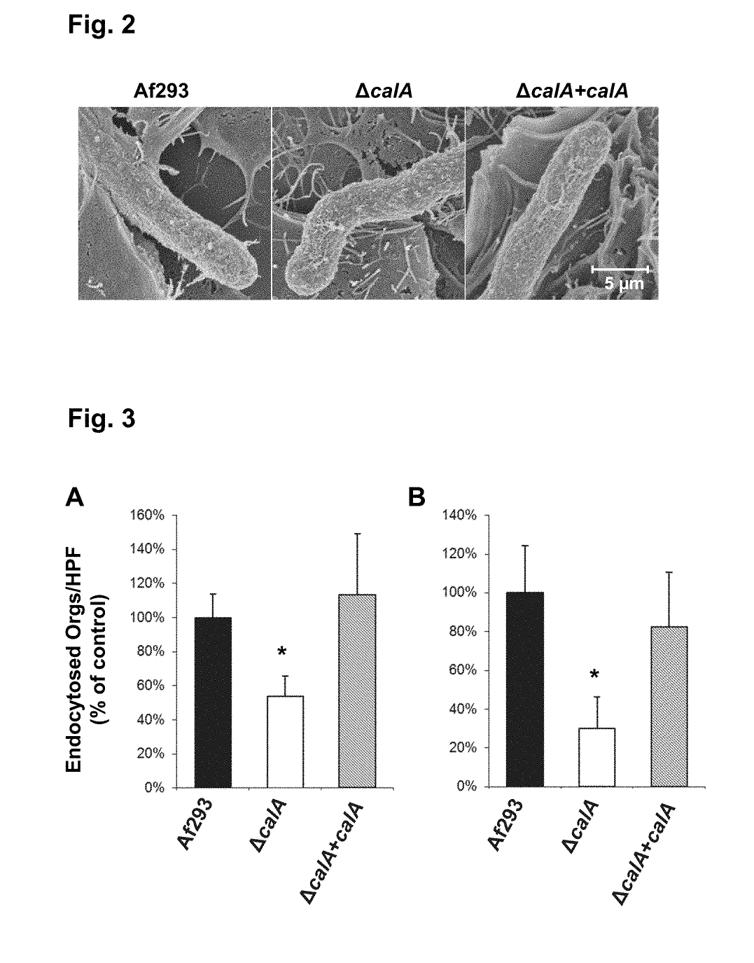

Further hyphal morphology changes by deletion of CalA were investigated. Conidia of three different strains were germinated in RPMI for 18 hours and then grown on A549 cells for 6 hours. Using scanning electron microscopy, we observed that the ΔCalA mutant had wiggly hyphal tips, which were significantly different from the straight hyphal tips formed by the control strains (FIG. 2). Interestingly, this phenotypic change only occurred when interacting with A549 cells, but not growing on the glass cover slips and positive or negative charge coated plastic surface (data not shown). The result indicates that CalA is required for normal hyphal development.

CalA is Dispensable for A. fumigatus Adherence to A549 Cells and Laminin Protein

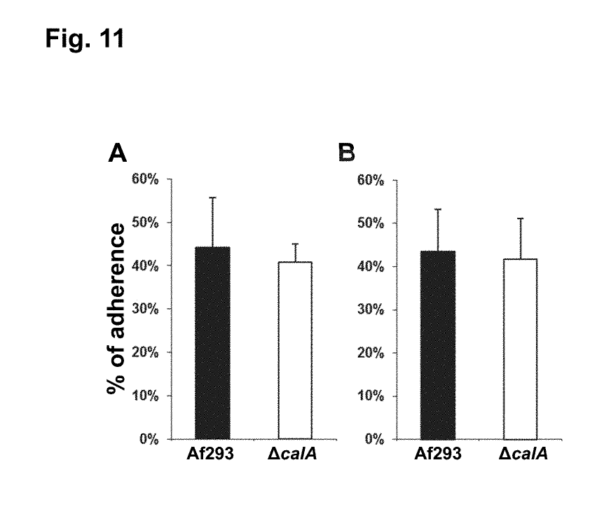

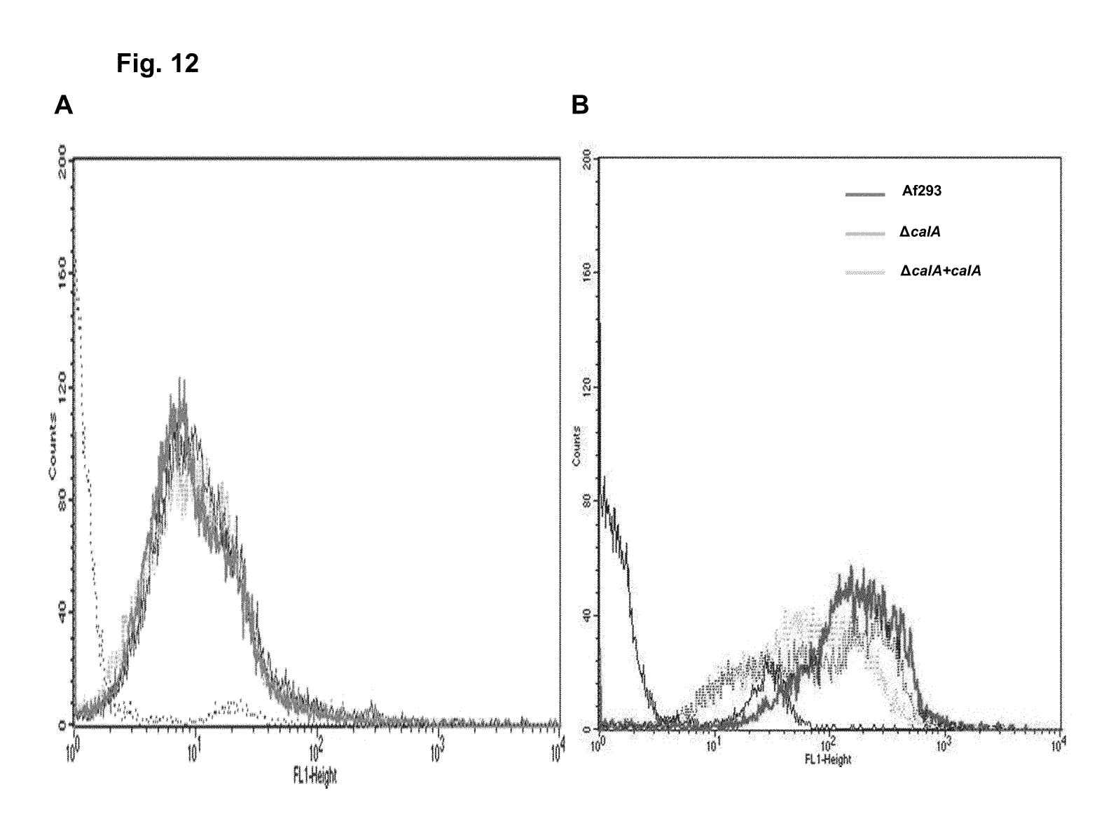

CalA is predicted to be an adhesin and it also binds to murine lung cells and the extracellular matrix protein laminin (Upadhyay et al., 2009). We hypothesized that CalA might mediate adherence to, and interactions with host cells. Therefore, we tested the capability of CalA deletion strain of A. fumigatus to adhere to A549 epithelial cells and laminin protein. By our standard adherence assay, we found that wild type and ΔCalA mutant had similar adherence to both A549 cells and laminin coated plates (FIG. 11A, B). Also, the flow cytometry results of different strains binding to fluorescence labeled laminin protein showed that deletion of CalA did not change the laminin binding capability of either swollen conidia or germlings (FIG. 12A, B). Therefore, these data suggest that CalA is dispensable for the adherence A. fumigatus.

### CalA is Required for Maximal A549 Cell Invasion

Epithelial cell invasion and damage occur following adherence of A. fumigatus to epithelial cells. We first investigate the role of CalA in mediating A549 cells invasion using our standard fluorescent assay in vitro. Conidia from different strains were pre-germinated in Sabroaud broth medium for 5.5 hour and then infected epithelial cells for 2.5 hours. We used Sabroaud broth medium, because in this medium ΔCalA mutant did not show any germination defect (data not shown). The endocytosis of the ΔCalA mutant was reduced by 47% compare to that of the wild-type strain (FIG. 3). And the ΔCalA mutant had 70% less invasion to the endothelial cells compared to the wild-type strain. The invasion defect was due to the absence of CalA because the ΔCalA+CalA complemented strain was endocytosed comparable to the wild-type strain.

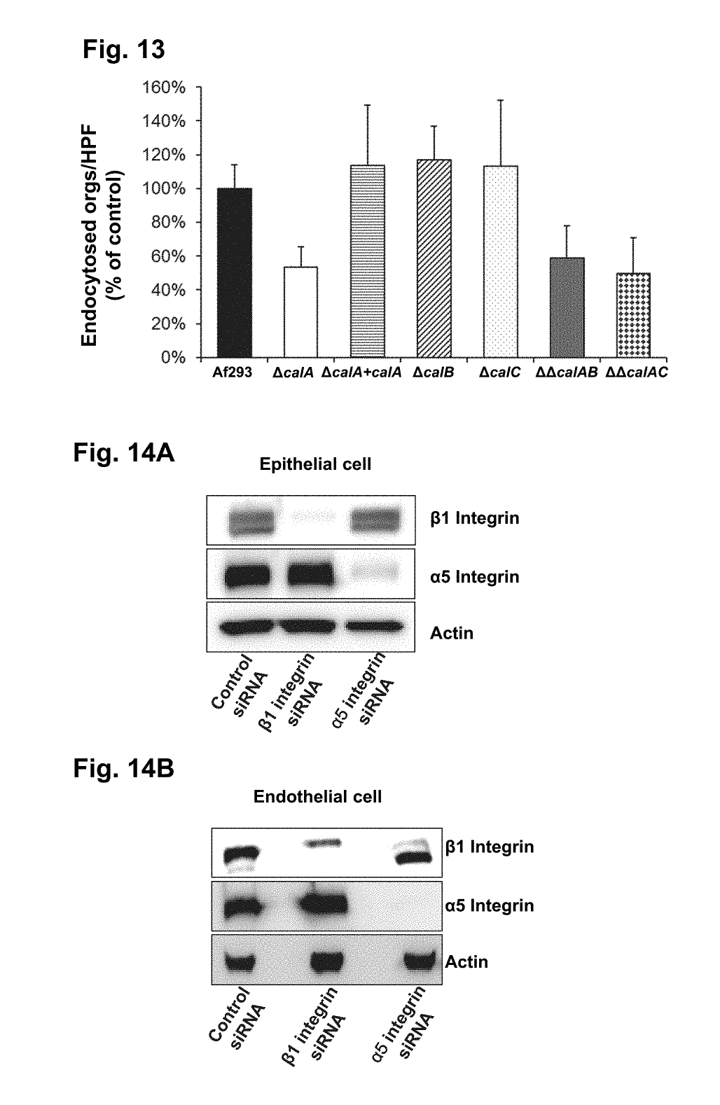

There are two orthologous genes of CalA in A. fumigatus, we named them CalB (Afu8g01710) and CalA (Afu3g00510). They shared 36% identity of the putative protein sequence. We also tested the invasion capacity of the single knock out strains of ΔCalB and ΔCalA, as well as the double deletion strains of ΔΔCalAB and ΔΔCalAC. We found even though they are orthologous genes, only CalA is required for maximal invasion of A. fumigatus (FIG. 13).

CalA-mCherry is Expressed in the Cell Surface of A. fumigatus

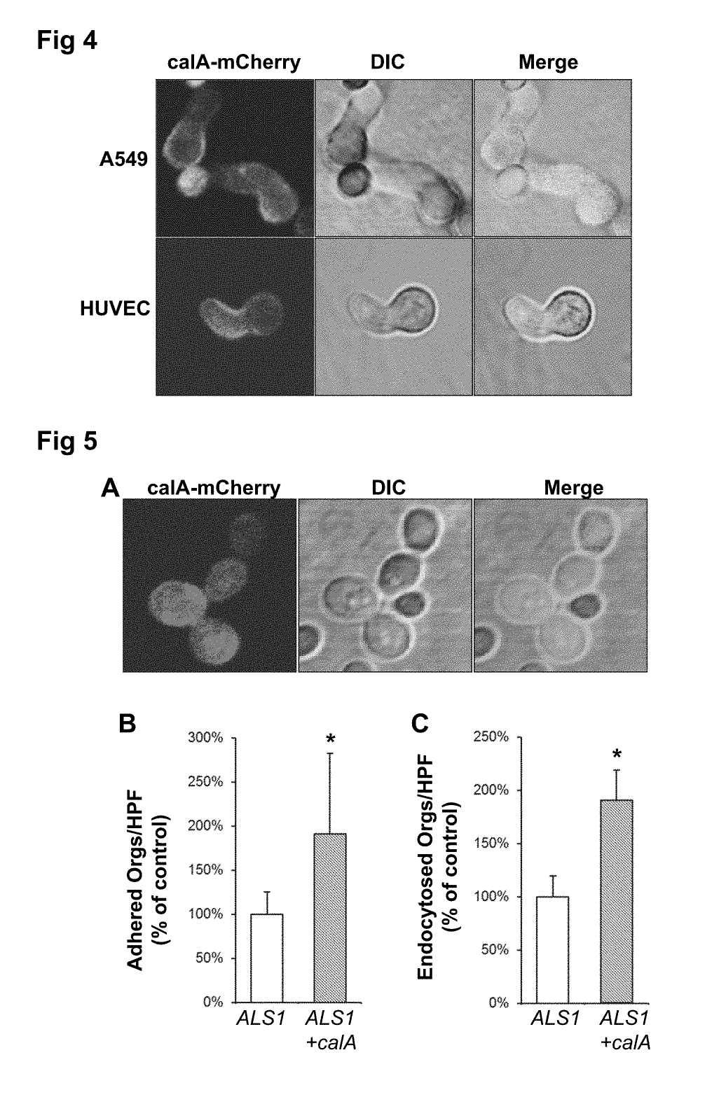

Various independent CalA-mCherry expressing strains were generated to analyze the expression pattern of CalA during epithelial cell and endothelial cell invasion by confocal microscopy. As shown in FIG. 4, the upper images were taken from invasion of epithelial cell and the bottom images were from invasion of the endothelial cell. We found that CalA was predominantly transcribed in cell surface.

CalA Functions as an Invasin after the Adherence has Occurred

To determine if CalA can directly mediate host cell invasion, we heterologously expressed it in a S. cerevisiae strain S150-2B. This S. cerevisiae strain does not adhere to or invade A549 cells and the endothelial cells. First we observed the CalA-mCherry expression in S. cerevisiae. Images showed that CalA was expressed throughout the cell, including the cell surface (FIG. 5A). Even though, this strain of S. cerevisiae expressed CalA on its surface, but it did not adhere to or invade A549 epithelial cells or endothelial cells.

The endocytosis occurs after the organism has adhered to host cells and in A. fumigatus. CalA is dispensable for adherence but it does necessary for maximal invasion to host cells. Then, we considered the possibility that CalA only induces host cell invasion after the adherence has occurred. To test this possibility, we expressed CalA-mCherry in a S. cerevisiae strains which also expressed Candida albicans Als1 (Fu et al., 1998). Als1 is a C. albicans adhesin, mediates adherence but induces very little invasion to human umbilical vein endothelial cells. The endothelial cell adherence of the S. cerevisiae strain expressing Als1 plus CalA increased by 50% compared to the control strain expressing Als1 alone (FIG. 5B). And the invasion of this strain to HUVECs increased by more than 100% (FIG. 5C). These data suggest that CalA functions as an invasin of A. fumigatus after the adherence has occurred.

### CalA Binds to β1 and α5 Integrins

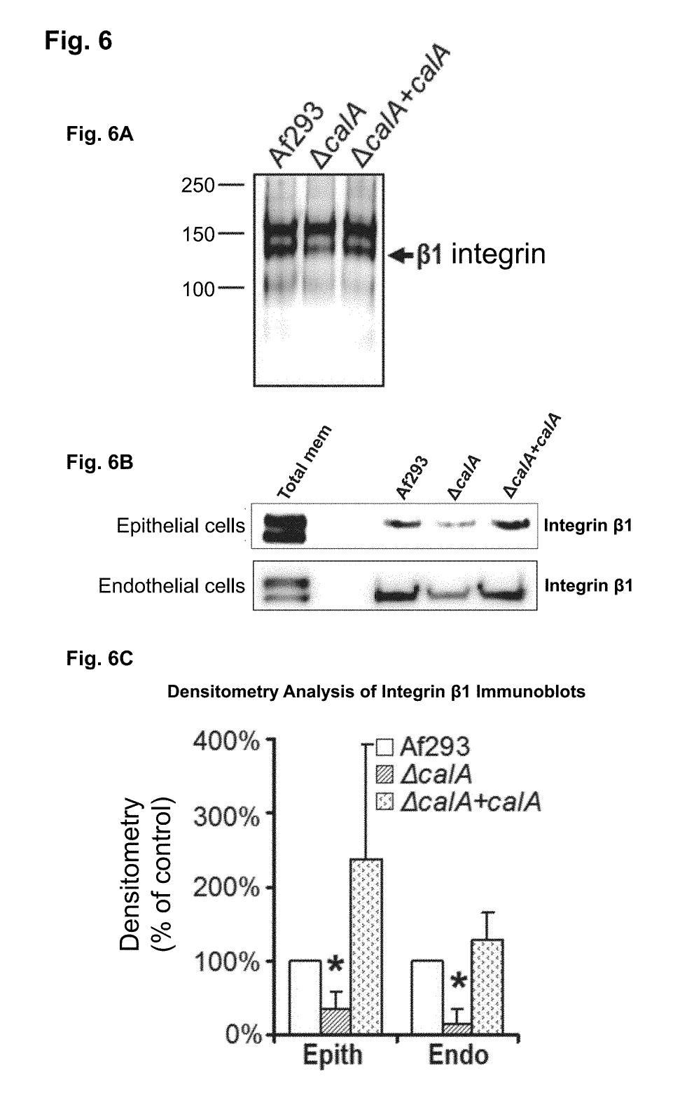

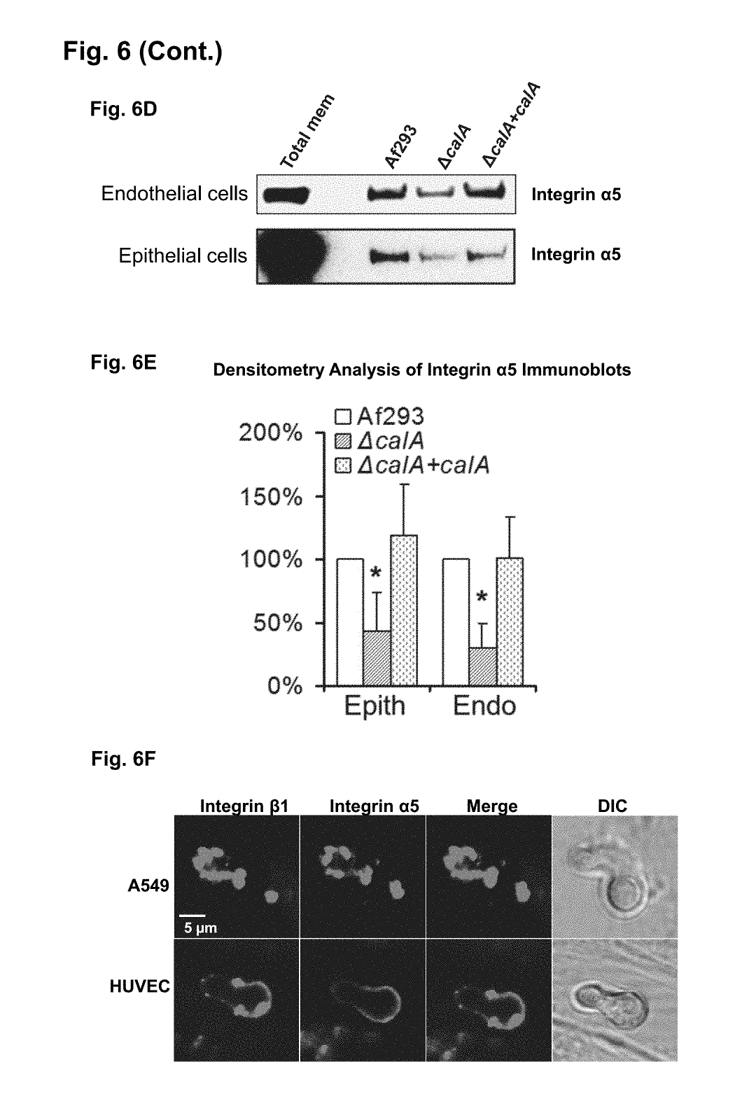

To identify the host cell receptors for A. fumigatus, the A549 epithelial cell membrane proteins were labeled by biotin, and extracted by 5.8% Octyl-glucopyranoside. The biotin labeled membrane proteins were affinity-purified over germ tubes of the wild-type A. fumigatus Af293, ΔcalA and ΔcalA+calA strains. The proteins bound to fungi were separated by SDS/PAGE and detected by immunoblotting with anti-biotin antibody (FIG. 6A). The wild-type strain and ΔcalA+calA strain bound to proteins with molecular masses of approximately 160, 130, and 100 kDa. The ΔcalA strain mutant bound to the 160 and 100 kDa proteins similarly to the wild-type and complemented strains, but bound less strongly to the 130 kDa protein. The band containing the 130 kDa protein was selected for protein sequencing by NanoLC-MS/MS which revealed the presence of β1 integrin.

To determine whether A. fumigatus interacts with integrin β1 in epithelial cells and endothelial cells, we performed immunoblots of cell membrane proteins of A549 epithelial cells or HUVEC vascular endothelial cells that were eluted from the three strains of A. fumigatus (wild type, ΔcalA and ΔcalA+calA). The proteins bound to fungi were separated by SDS/PAGE and detected by immunoblotting with anti-integrin β1 antibody (ab52971 Abcam). The results confirmed that integrin β1 from epithelial cells (FIG. 6B, top panel) and endothelial cells (FIG. 6B, bottom panel) binds strongly to wild type and ΔCalA+CalA strains of A. fumigatus and importantly determined that the ΔCalA mutant strain bound weakly to integrin β1 (FIG. 6B). Densitometric analysis of replicate immunoblots similar to those of FIG. 6B showed that the ΔCalA mutant bound 64% and 86% less integrin β1 in epithelial cells and endothelial cells, respectively, as compared to the wild-type strain (FIG. 6C). Thus, A. fumigatus interacts, either directly or indirectly, with integrin β1, and this interaction is largely dependent on CalA.

The integrin β subunit and α subunit can form heterodimers and functions as a complex (Giancotti and Ruoslahti, 1999). To determine whether A. fumigatus interacts with integrin α on epithelial cells and endothelial cells, we performed immunoblots of cell membrane proteins of A549 epithelial cells or HUVEC vascular endothelial cells that were eluted from the three strains of A. fumigatus (wild type, ΔcalA and ΔcalA+calA). The proteins bound to fungi were separated by SDS/PAGE and detected by immunoblotting with an anti-integrin α5 antibody (ab150361 Abcam). The results confirmed that integrin α5 from endothelial cells (FIG. 6D, top panel) and epithelial cells (FIG. 6D, bottom panel) binds strongly to wild type and ΔCalA+CalA strains of A. fumigatus and importantly determined that the ΔCalA mutant strain bound weakly to integrin α5 (FIG. 6D). Densitometric analysis of replicate immunoblots similar to those of FIG. 6D showed that the ΔCalA mutant bound significantly less integrin α5 in epithelial cells and endothelial cells as compared to the wild-type strain (FIG. 6E). Thus, A. fumigatus interacts, either directly or indirectly, with integrin α5, and this interaction is also largely dependent on CalA.

To determine the binding of A. fumigatus to protein integrin α5β1 upon endocytosis, we infected epithelial cell or endothelial cell with A. fumigatus and examined the distribution of integrin β1 and α5 by confocal microscopy. We observed that integrin β1 and α5 accumulated together around the same hyphal or conidial surface of A. fumigatus germlings (FIG. 6F). The above results strongly suggest that A. fumigatus interacts with integrin α5β1 in both intact epithelial cells and endothelial cells during endocytosis and this interaction is mediated by CalA.

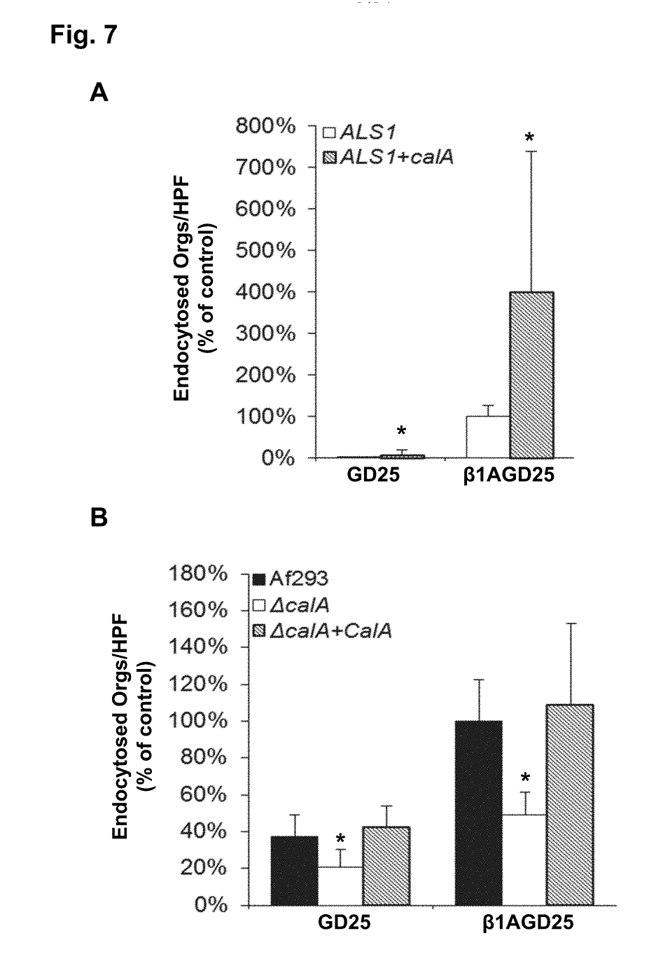

To determine the function of integrin β1 in mediating Aspergillus endocytosis, we compared GD25 the integrin β1 knock out cell line and β1AGD25 the integrin β1 restored cell line. First, we compared the endocytosis of S. ceravisia with or without expression of CalA. We found both strains were rarely taken up by GD25, but significant endocytosis occurred by β1AGD25 cells. In addition, expressing CalA increased 3 times endocytosis when compared to control (FIG. 7A). Then, we tested the Af293, ΔCalA mutant and complemented stains using the two cell lines. Results showed that even though all three strains can be endocytosed by the two cell lines, the endocytosis in β1AGD25 cells was much higher, and knocking out CalA reduced host cell endocytosis in both cell lines (FIG. 7B). These data indicate that interaction of CalA and integrin β1 is required for host cell endocytosis.

To determine if integrin α works functionally together with β1, we screened different α integrins by antibody blocking with anti-α integrin antibodies. The anti-α5 integrin antibody was identified to have similar inhibition effects as anti-β1 integrin antibody in both epithelial and endothelial cells (FIG. 8A, B). Since the A549 epithelial cell line was type II alveolar epithelial cells, we also tested antibody blocking endocytosis by human primary pulmonary alveolar epithelial cells which contains 95% type I epithelial cells. Result showed that antibody blocking function of β1 integrin (i.e., integrin β1) or α5 integrin (i.e., integrin α5) inhibited about 50% endocytosis in primary epithelial cells (FIG. 8C), which was the same as using the A549 cell line.

Furthermore, we confirmed the function of integrin α5β1 in mediating host cell endocytosis by siRNA knockdown assay. The effect of siRNA knockdown on integrin β1 or α5 was shown in FIG. 14. and FIG. 8 D-E which indicated that knockdown of either integrin β1 or α5 significantly inhibited endocytosis in epithelial cell and endothelial cell. These results suggest the α5 subunit works together with integrin β1 to mediate endocytosis.

CalA is Required for Full Virulence of A. fumigatus

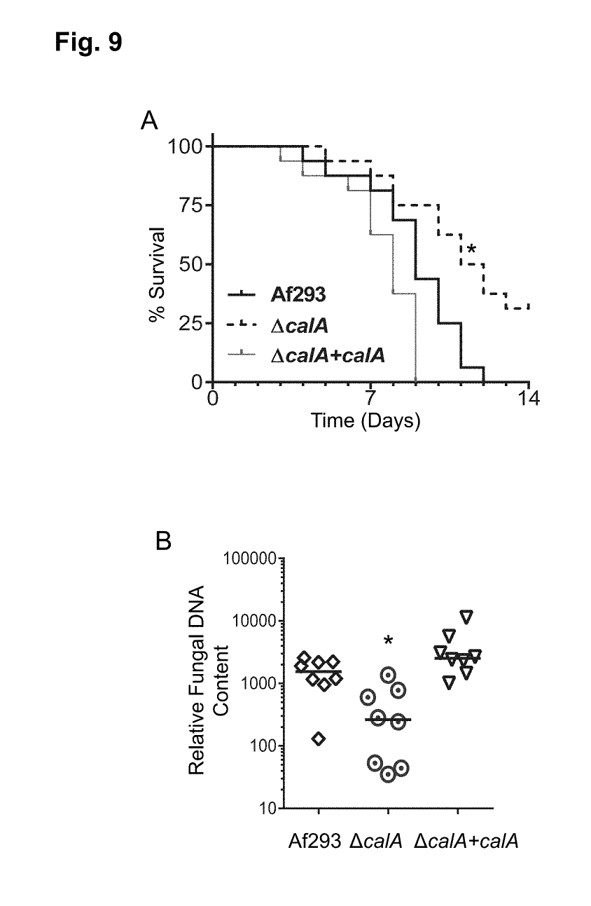

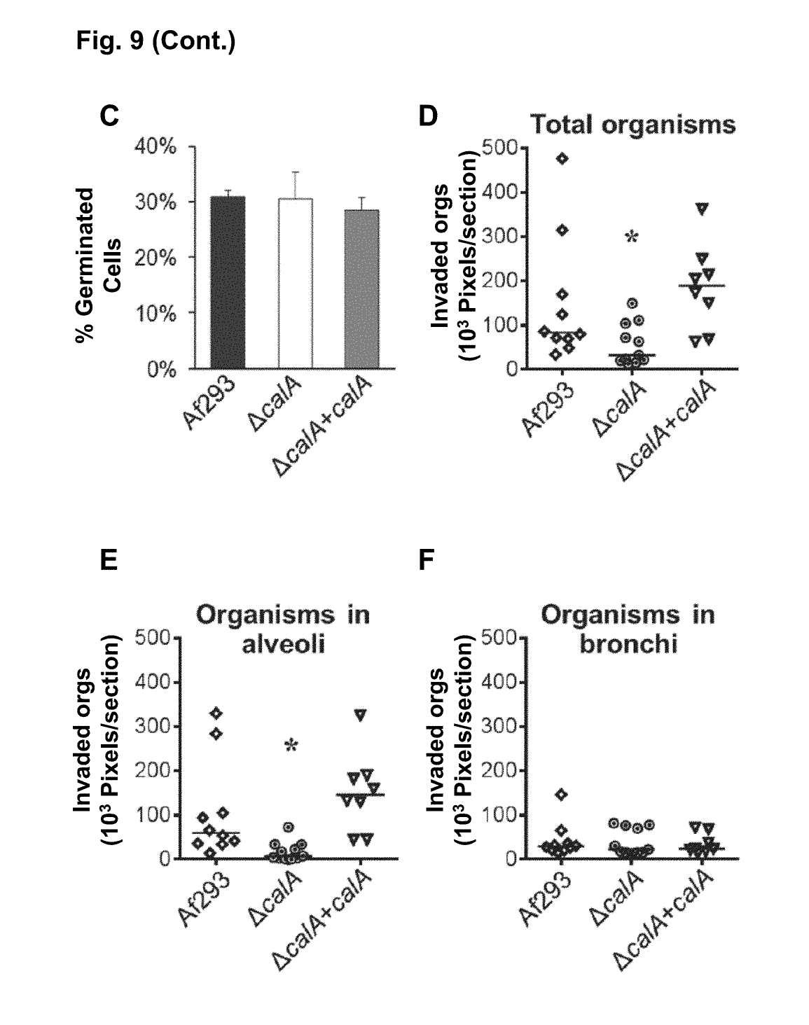

The identified function of CalA in mediating host cell invasion and damage in our studies suggested that CalA may contribute to A. fumigatus virulence. To test this hypothesis, we determined the virulence of the ΔCalA mutant in a murine model of invasive pulmonary aspergillosis. The mice were immunosuppressed with five doses of cortisone acetate. The immunosuppressed mice were infected by placing them in an acrylic chamber filled with an aerosol of different strains of A. fumigatus conidia. We found that mice infected with ΔCalA mutant had better survival curve compare to that of the wild-type and ΔCalA+CalA complemented strains (FIG. 9A). Furthermore, mice lungs infected by ΔCalA mutant had about 6 times lower fungal burden compared to wild type and complemented strains, at day 4 after infection by real-time PCR detection (FIG. 9B). These results indicate that CalA is required for maximal virulence in murine model of invasive pulmonary aspergillosis.

Inhibition of CalA by an Anti-CalA Antibody Increase the Survive of Mouse from Invasive Aspergillosis

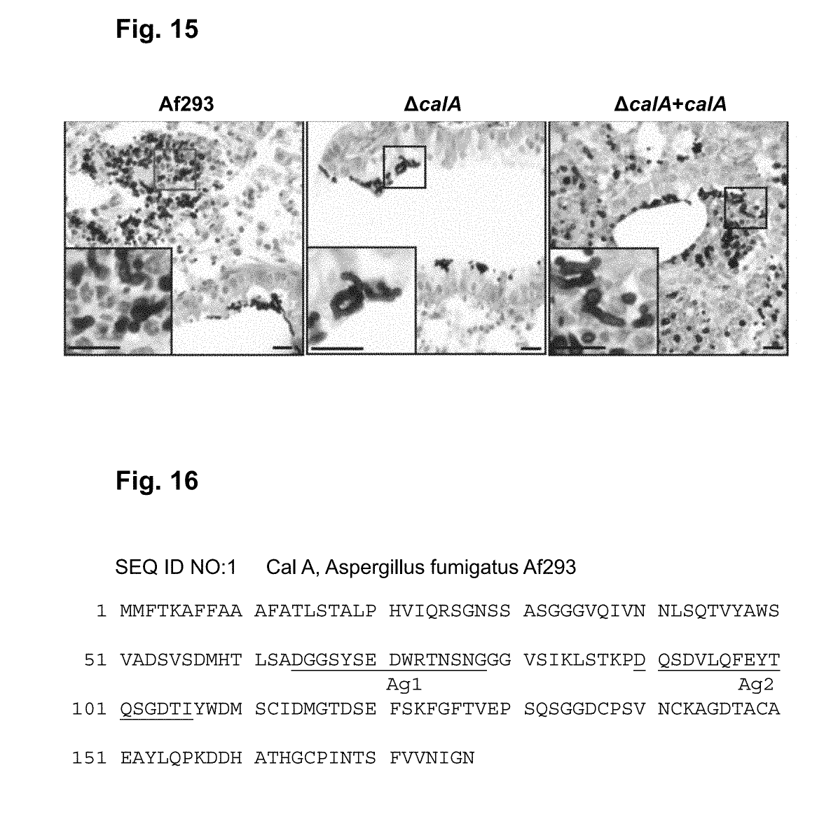

Disruption of CalA in A. fumigatus significantly increased the chance of the mice surviving from invasive aspergillosis. However, the ΔCalA mutant has a germination delay when contacts with epithelial cells and a decreased invasion to host cells. To ascertain the attenuated virulence of this mutant is due to host cell invasion defect, we treated mice by intratracheal infection with 10⁷ conidia per mouse. We observed germination of each strain in mouse lungs by histopathological examination at 12 h after infection. Images show that the ΔCalA mutant has wild type germination rate in vivo (FIG. 15). Therefore, the virulence defect of ΔCalA mutant is due to reduced invasion to host cells.

Because CalA is a cell surface protein, it could be a target for antifungal therapy of invasive aspergillosis. To test this hypothesis, we produced an anti-CalA antibody (Anti-CalA2), which was raised against a peptide (Ag2) that comprised a predicted cell surface domain of CalA. First, we determined that the Anti-CalA2 antibody blocked host cell invasion of A. fumigatus in both epithelial cell and endothelial cells (FIG. 10A, B). Next, we treated mice with rabbit control IgG or Anti-CalA2 antibody and compared the survival of the two groups. We found that mice treated with Anti-CalA2 antibody significantly increased the chance of surviving from invasive aspergillosis (FIG. 10C). The effectiveness of Anti-CalA2 was further demonstrated by the finding that antibodies raised against Ag1 (e.g., Anti-CalA1) did not block invasion of A549 cells and were not protective in mice. These data indicate that CalA, and specifically the polypeptide region of Ag2, is a viable target for antifungal strategies to treat or prevent invasive aspergillosis.

TABLE 2
A. fumigatus and S. Cerevisiae used herein.

| Strain | Relevant Genotype | Reference |
|---|---|---|
| A. fumigauts | | |
| Af293 | Wild-type clinical isolate | ATCC |
| CEA10 | Wild-type clinical isolate | ¹ |
| ATCC46645 | Wild-type clinical isolate | ² |
| CalA-RFP | CalA-RFP::ble* | This study |
| ΔcalA | ΔcalA::hph* | This study |
| ΔcalA + calA | ΔcalA::hph; pcalA* | This study |
| Af293-GFP | GFP* | This study |
| ΔcalA-GFP | ΔcalA::hph; GFP* | This study |
| ΔcalA + calA-GFP | ΔcalA::hph; pcalA; GFP* | This study |
| S. cerevisiae | | |
| S150-2B | MATa leu⁻, 112 ura ⁻, trp⁻, his3⁻ | ³ |
| CalA-S | pcalA-RFP^{†} | This study |
| pESC-S | pESC^{†} | This study |
| CalAAls1 | pcalA-RFP; pALS1^{†} | This study |
| pESCAls1 | pESC; pALS1^{†} | This study |

*Strains are in Af293 background,
^{†}strains are in S150-2B background.
¹ Hearn, V. M., Wilson, E. V. & Mackenzie, D. W. The preparation of protoplasts from Aspergillus fumigatus mycelium. Sabouraudia 18, 75-7 (1980).
² Monod, M. et al. Virulence of alkaline protease-deficient mutants of Aspergillus fumigatus. FEMS Microbiol Lett 106, 39-46 (1993).
³ Fu, Y. et al. Expression of the Candida albicans gene ALS1 in Saccharomyces cerevisiae induces adherence to endothelial and epithelial cells. Infect Immun 66, 1783-6 (1998).

### REFERENCES

Belaish, R., Sharon, H., Levdansky, E., Greenstein, S., Shadkchan, Y., and Osherov, N. (2008) The Aspergillus nidulans cetA and CalA genes are involved in conidial germination and cell wall morphogenesis. Fungal Genet Biol 45: 232-242.
Bouchara, J. P., Sanchez, M., Chevailler, A., Marot-Leblond, A., Lissitzky, J. C., Tronchin, G., and Chabasse, D. (1997) Sialic acid-dependent recognition of laminin and fibrinogen by Aspergillus fumigatus conidia. Infect Immun 65: 2717-2724.
Catlett, N. L., Lee, B., Yoder, O. C., and Turgeon, B. G. (2002) Split-Marker Recombination for Efficient Targeted Deletion of Fungal Genes. In Fungal Genet. Vol. News., pp. 9-11.
Ejzykowicz, D. E., Cunha, M. M., Rozental, S., Solis, N. V., Gravelat, F. N., Sheppard, D. C., and Filler, S. G. (2009) The Aspergillus fumigatus transcription factor Ace2 governs pigment production, conidiation and virulence. Mol Microbiol 72: 155-169.
Filler, S. G., and Sheppard, D. C. (2006) Fungal invasion of normally non-phagocytic host cells. PLoS Pathog 2: e129.
Fu, Y., Rieg, G., Fonzi, W. A., Belanger, P. H., Edwards, J. E., Jr., and Filler, S. G. (1998) Expression of the Candida albicans gene ALS1 in Saccharomyces cerevisiae induces adherence to endothelial and epithelial cells. Infect Immun 66: 1783-1786.
Giancotti, F. G., and Ruoslahti, E. (1999) Integrin signaling. Science 285: 1028-1032.
Gravelat, F. N., Askew, D. S., and Sheppard, D. C. (2012) Targeted gene deletion in Aspergillus fumigatus using the hygromycin-resistance split-marker approach. Methods Mol Biol 845: 119-130.
Gravelat, F. N., Beauvais, A., Liu, H., Lee, M. J., Snarr, B. D., Chen, D., Xu, W., Kravtsov, I., Hoareau, C. M., Vanier, G., Urb, M., Campoli, P., Al Abdallah, Q., Lehoux, M., Chabot, J. C., Ouimet, M. C., Baptista, S. D., Fritz, J. H., Nierman, W. C., Latge, J. P., Mitchell, A. P., Filler, S. G., Fontaine, T., and Sheppard, D. C. (2013) Aspergillus galactosaminogalactan mediates adherence to host constituents and conceals hyphal beta-glucan from the immune system. PLoS Pathog 9: e1003575.
Gravelat, F. N., Ejzykowicz, D. E., Chiang, L. Y., Chabot, J. C., Urb, M., Macdonald, K. D., al-Bader, N., Filler, S. G., and Sheppard, D. C. (2010) Aspergillus fumigatus MedA governs adherence, host cell interactions and virulence. Cell Microbiol 12: 473-488.
Kousha, M., Tadi, R., and Soubani, A. O. (2011) Pulmonary aspergillosis: a clinical review. Eur Respir Rev 20: 156-174.
Liu, H., Gravelat, F. N., Chiang, L. Y., Chen, D., Vanier, G., Ejzykowicz, D. E., Ibrahim, A. S., Nierman, W. C., Sheppard, D. C., and Filler, S. G. Aspergillus fumigatus AcuM regulates both iron acquisition and gluconeogenesis. Mol Microbiol 78: 1038-1054.
Lopes Bezerra, L. M., and Filler, S. G. (2004) Interactions of Aspergillus fumigatus with endothelial cells: internalization, injury, and stimulation of tissue factor activity. Blood 103: 2143-2149.
Marr, K. A., Carter, R. A., Crippa, F., Wald, A., and Corey, L. (2002) Epidemiology and outcome of mould infections in hematopoietic stem cell transplant recipients. Clin Infect Dis 34: 909-917.
Maschmeyer, G., Haas, A., and Cornely, O. A. (2007) Invasive aspergillosis: epidemiology, diagnosis and management in immunocompromised patients. Drugs 67: 1567-1601.
Patterson, T. F., Kirkpatrick, W. R., White, M., Hiemenz, J. W., Wingard, J. R., Dupont, B., Rinaldi, M. G., Stevens, D. A., and Graybill, J. R. (2000) Invasive aspergillosis. Disease spectrum, treatment practices, and outcomes. I3 Aspergillus Study Group. Medicine (Baltimore) 79: 250-260.
Patterson, T. F., Boucher, H. W., Herbrecht, R., Denning, D. W., Lortholary, O., Ribaud, P., Rubin, R. H., Wingard, J. R., DePauw, B., Schlamm, H. T., Troke, P., and Bennett, J. E. (2005) Strategy of following voriconazole versus amphotericin B therapy with other licensed antifungal therapy for primary treatment of invasive aspergillosis: impact of other therapies on outcome. Clin Infect Dis 41: 1448-1452.
Pegues, C. F., Daar, E. S., and Murthy, A. R. (2001) The epidemiology of invasive pulmonary aspergillosis at a large teaching hospital. Infect Control Hosp Epidemiol 22: 370-374.
Phan, Q. T., Fratti, R. A., Prasadarao, N. V., Edwards, J. E., Jr., and Filler, S. G. (2005) N-cadherin mediates endocytosis of Candida albicans by endothelial cells. J Biol Chem 280: 10455-10461.
Phan, Q. T., Myers, C. L., Fu, Y., Sheppard, D. C., Yeaman, M. R., Welch, W. H., Ibrahim, A. S., Edwards, J. E., Jr., and Filler, S. G. (2007) Als3 is a Candida albicans invasin that binds to cadherins and induces endocytosis by host cells. PLoS Biol 5: e64.
Richie, D. L., Miley, M. D., Bhabhra, R., Robson, G. D., Rhodes, J. C., and Askew, D. S. (2007) The Aspergillus fumigatus metacaspases CasA and CasB facilitate growth under conditions of endoplasmic reticulum stress. Mol Microbiol 63: 591-604.
Sheppard, D. C., Yeaman, M. R., Welch, W. H., Phan, Q. T., Fu, Y., Ibrahim, A. S., Filler, S. G., Zhang, M., Waring, A. J., and Edwards, J. E., Jr. (2004) Functional and structural diversity in the Als protein family of Candida albicans. J Biol Chem 279: 30480-30489.
Upadhyay, S. K., Mahajan, L., Ramjee, S., Singh, Y., Basir, S. F., and Madan, T. (2009) Identification and characterization of a laminin-binding protein of Aspergillus fumigatus: extracellular thaumatin domain protein (AfCalAp). J Med Microbiol 58: 714-722.
Williamson, E. C., Leeming, J. P., Palmer, H. M., Steward, C. G., Warnock, D., Marks, D. I., and Millar, M. R. (2000) Diagnosis of invasive aspergillosis in bone marrow transplant recipients by polymerase chain reaction. Br J Haematol 108: 132-139.

### Example 2

Non-limiting examples of polypeptide sequences.

SEQ ID NO.: 1
Cal A, Aspergillus fumigatus (Af293)
MMFTKAFFAAAFATLSTALPHVIQRSGNSSASGGGVQIVNNLSQTVYAWS
VADSVSDMHTLSADGGSYSEDWRTNSNGGGVSIKLSTKPDQSDVLQFEYT
QSGDTIYWDMSCIDMGTDSEFSKFGFTVEPSQSGGDCPSVNCKAGDTACA
EAYLQPKDDHATHGCPINTSFVVNIGN
SEQ ID NO.: 2
Antigen 1 (derived from Aspergillus fumigatus)
LSADGGSYSEDWRTNSNG
SEQ ID NO.: 3
Antigen 2 (derived from Aspergillus fumigatus)
DQSDVLQFEYTQSGDTI
SEQ ID NO.: 4
Antigen 3 (derived from Aspergillus fumigatus)
QSDVLQFEYTQSGDTI
SEQ ID NO.: 5
Antigen 4 (derived from P. digitatum)
NQQDVLQFEYTEAGDTI
SEQ ID NO.: 6
Antigen 5 (derived from A. nidulans)
DQTDVLQFEYTKSGETI
SEQ ID NO.: 7
Antigen 6 (derived from A. terreus)
TQADVLQFEYTEAGDTI
SEQ ID NO.: 8
Antigen 7 (derived from A. kawachii)
SQSDVLQFEYTQSGDTI
SEQ ID NO.: 9
Antigen 8 (derived from A. niger)
SQSDVLQFEYTQDGDTI
SEQ ID NO.: 10
Antigen 9 (derived from A. flavus and A. oryzae)
EQSNVLQFEYTQSGDTI
SEQ ID NO.: 11
Antigen 10 (derived from A. ruber)
NQDDVLQFEYTQSGDTI
SEQ ID NO.: 12
Antigen 11 (derived from A. clavatus)
DQSDVLQFEYTQSDQTI
SEQ ID NO.: 13
Antigen 12 (derived from A. fumigatus and A.
fischeri)
DQSDVLQFEYTQSGDTI
SEQ ID NO.: 14
Consensus 1
QX₁X₂VLQFEYTX₃SGX₄TI
where X₁ is S or T; X₂ is D or N; X₃ is Q or K;
and X₄ is D, E or Q.
SEQ ID NO.: 15
Consensus 2
X₁QX₂X₃VLQFEYTX₄X₅X₆X7TI
where X₁ is D, N, T, E or S; X₂ is D, S, A, T or
Q; X₃ is D or N; X₄ is Q, E or K; X₅ is S, D
or A; X₆ is G or D; and X7 is D, E or Q.
SEQ ID NO.: 16
Consensus 3
VLQFEYT

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The term “about” as used herein means any value that is within 10%, higher or lower, of an indicated value.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It will be understood by those of skill in the art that numerous and various modifications can be made without departing from the spirit of the technology. Therefore, it should be clearly understood that the forms of the technology are illustrative only and are not intended to limit the scope of the technology.

1. A pharmaceutical composition comprising:
a CalA binding agent; and
a pharmaceutical acceptable excipient, diluent, additive or carrier,
wherein the binding agent specifically binds to a CalA polypeptide.

2. The pharmaceutical composition of claim 1, wherein the binding agent comprises an antibody, or a binding fragment thereof.

3. (canceled)

4. The pharmaceutical composition of claim 2, wherein the antibody is a monoclonal antibody, or binding fragment thereof.

5. (canceled)

6. The pharmaceutical composition of claim 4, wherein the monoclonal antibody is a chimeric antibody or humanized antibody.

7-10. (canceled)

11. The pharmaceutical composition of claim 1, wherein the CalA polypeptide comprises a polypeptide having at least 76%, or at least 80% identity to the amino acid sequence of Antigen 2 (SEQ ID NO: 47).

12. (canceled)

13. The pharmaceutical composition of claim 11, wherein the CalA polypeptide comprises a polypeptide having the amino acid sequence represented by the formula, X₁QX₂X₃VLQFEYTX₄X₅X₆X₇TI (SEQ ID NO.: 15),
where X1 is D, N, T, E or S; X2 is D, S, A, T or Q; X3 is D or N; X4 is Q, E, or K; X5 is S, D or A; X6 is G or D; and X7 is D, E or Q.

14. The pharmaceutical composition of claim 1, wherein the CalA polypeptide comprises the amino acid sequence of Antigen 2 (SEQ ID NO: 47).

15. The pharmaceutical composition of claim 1, wherein the CalA polypeptide comprises the amino acid sequence VLQFEYT (SEQ ID NO.: 16).

16. The pharmaceutical composition of claim 1, wherein the CalA polypeptide is a wild type CalA protein expressed by the genus Aspergillus, Penicillium or Neosartorya.

17. The pharmaceutical composition of claim 16, wherein the CalA polypeptide is a wild type CalA protein expressed by one of A. fumigatus, A. kawachii, A. flavus, A. oryzae, A. ruber, A. niger, A. clavatus, and A. nidulans.

18. (canceled)

19. The pharmaceutical composition of claim 1, wherein the pharmaceutical composition comprises one or more antifungal medications configured for administration to a mammal.

20. The pharmaceutical composition of claim 19, wherein the antifungal medication comprises a polyene antimycotic, an imidazole antifungal medication, a triazole antifungal medication, an abafungin, an allylamine antifungal medication, or an echinocandin antifungal medication.

21-31. (canceled)

32. The pharmaceutical composition of claim 19, wherein the antifungal medication is selected from one or more of amphotericin B, anidulafungin, caspofungin, fluconazole, flucytosine, micafungin, posaconazole, and voriconazole.

33. (canceled)

34. The pharmaceutical composition of claim 1, wherein the pharmaceutical composition is substantially free of serum proteins and is sterile.

35. (canceled)

36. The pharmaceutical composition of claim 1, wherein the pharmaceutical composition comprises a sterile, lyophilized powder suitable for intravenous administration to a mammal.

37. A method of administering to a subject in need thereof, a therapeutically effective amount of the pharmaceutical composition of claim 1.

38-73. (canceled)

74. The method of claim 37, wherein the subject in need thereof is a human who has, or is at risk of acquiring, a fungal infection.

75-76. (canceled)

77. A pharmaceutical composition comprising:
a CalA polypeptide; and
an adjuvant,
wherein the CalA polypeptide comprises an amino acid sequence having at least 80% identity to the amino acid sequence of Antigen 2 (SEQ ID NO: 47).

78-113. (canceled)

114. A method comprising:
administering to a subject in need thereof, a therapeutically effective amount of the pharmaceutical composition of claim 77.

115-148. (canceled)


SEQUENCE LISTING (59 sequences)

SEQ ID NO: 1 - protein, 177 aa, Aspergillus fumigatus
MMFTKAFFAAAFATLSTALPHVIQRSGNSSASGGGVQIVNNLSQTVYAWSVADSVSDMHTLSADGGSYSEDWRTNSNGGGVSIKLSTKPDQSDVLQFEYTQSGDTIYWDMSCIDMGTDSEFSKFGFTVEPSQSGGDCPSVNCKAGDTACAEAYLQPKDDHATHGCPINTSFVVNIGN

SEQ ID NO: 2 - protein, 15 aa, Artificial Sequence
  Description of Artificial Sequence CalA Polypeptide
DGGSYSEDWRTNSNG

SEQ ID NO: 3 - protein, 17 aa, Artificial Sequence
  Description of Artificial Sequence CalA Polypeptide
DQSDVLQFEYTQSGDTI

SEQ ID NO: 4 - protein, 16 aa, Artificial Sequence
  Description of Artificial Sequence Antigen 3
QSDVLQFEYTQSGDTI

SEQ ID NO: 5 - protein, 17 aa, Artificial Sequence
  Description of Artificial Sequence Antigen 4
NQQDVLQFEYTEAGDTI

SEQ ID NO: 6 - protein, 17 aa, Artificial Sequence
  Description of Artificial Sequence Antigen 5
DQTDVLQFEYTKSGETI

SEQ ID NO: 7 - protein, 17 aa, Artificial Sequence
  Description of Artificial Sequence Antigen 6
TQADVLQFEYTEAGDTI

SEQ ID NO: 8 - protein, 17 aa, Artificial Sequence
  Description of Artificial Sequence Antigen 7
SQSDVLQFEYTQSGDTI

SEQ ID NO: 9 - protein, 17 aa, Artificial Sequence
  Description of Artificial Sequence Antigen 8
SQSDVLQFEYTQDGDTI

SEQ ID NO: 10 - protein, 17 aa, Artificial Sequence
  Description of Artificial Sequence Antigen 9
EQSNVLQFEYTQSGDTI

SEQ ID NO: 11 - protein, 17 aa, Artificial Sequence
  Description of Artificial Sequence Antigen 10
NQDDVLQFEYTQSGDTI

SEQ ID NO: 12 - protein, 17 aa, Artificial Sequence
  Description of Artificial Sequence Antigen 11
DQSDVLQFEYTQSDQTI

SEQ ID NO: 13 - protein, 17 aa, Artificial Sequence
  Description of Artificial Sequence Antigen 12
DQSDVLQFEYTQSGDTI

SEQ ID NO: 14 - protein, 16 aa, Artificial Sequence
  Description of Artificial Sequence CalA Polypeptide
  SITE (2)..(2): Xaa is S or T
  SITE (3)..(3): Xaa is D or N
  SITE (11)..(11): Xaa is Q or K
  SITE (14)..(14): Xaa is D, E or Q
QXXVLQFEYTXSGXTI

SEQ ID NO: 15 - protein, 17 aa, Artificial Sequence
  Description of Artificial Sequence CalA Polypeptide
  SITE (1)..(1): Xaa is D, N, T, E or S
  SITE (3)..(3): Xaa is D, S, A, T or Q
  SITE (4)..(4): Xaa is D or N
  SITE (12)..(12): Xaa is Q, E or K
  SITE (13)..(13): Xaa is S, D or A
  SITE (14)..(14): Xaa is G or D
  SITE (15)..(15): Xaa is D, E or Q
XQXXVLQFEYTXXXXTI

SEQ ID NO: 16 - protein, 7 aa, Artificial Sequence
  Description of Artificial Sequence CalA Polypeptide
VLQFEYT

SEQ ID NO: 17 - DNA, 23 bp, Artificial Sequence
  Description of Artificial Sequence Primer
caccagtacgtcggtcaatcttg

SEQ ID NO: 18 - DNA, 19 bp, Artificial Sequence
  Description of Artificial Sequence Primer
actgaggatgtagagactg

SEQ ID NO: 19 - DNA, 22 bp, Artificial Sequence
  Description of Artificial Sequence Primer
cacctcaactcttgcttgatgc

SEQ ID NO: 20 - DNA, 19 bp, Artificial Sequence
  Description of Artificial Sequence Primer
actgcttctgcatcatcag

SEQ ID NO: 21 - DNA, 20 bp, Artificial Sequence
  Description of Artificial Sequence Primer
cacctggagtggtacatgcg

SEQ ID NO: 22 - DNA, 18 bp, Artificial Sequence
  Description of Artificial Sequence Primer
agtcatgactcatacggc

SEQ ID NO: 23 - DNA, 22 bp, Artificial Sequence
  Description of Artificial Sequence Primer
caccagtctgatctacagagag

SEQ ID NO: 24 - DNA, 18 bp, Artificial Sequence
  Description of Artificial Sequence Primer
tgagttctagtccatggc

SEQ ID NO: 25 - DNA, 23 bp, Artificial Sequence
  Description of Artificial Sequence Primer
caccacgtgagtactgatgagtg

SEQ ID NO: 26 - DNA, 18 bp, Artificial Sequence
  Description of Artificial Sequence Primer
agactgtagcacaatggc

SEQ ID NO: 27 - DNA, 21 bp, Artificial Sequence
  Description of Artificial Sequence Primer
caccgtgaggtacagctctgc

SEQ ID NO: 28 - DNA, 18 bp, Artificial Sequence
  Description of Artificial Sequence Primer
aggcagtctcgagcatgg

SEQ ID NO: 29 - DNA, 20 bp, Artificial Sequence
  Description of Artificial Sequence Primer
ggatgcctccgctcgaagta

SEQ ID NO: 30 - DNA, 20 bp, Artificial Sequence
  Description of Artificial Sequence Primer
cgttgcaagacctgcctgaa

SEQ ID NO: 31 - DNA, 19 bp, Artificial Sequence
  Description of Artificial Sequence Primer
tgatgaacagggtcacgtc

SEQ ID NO: 32 - DNA, 19 bp, Artificial Sequence
  Description of Artificial Sequence Primer
aagttgaccagtgccgttc

SEQ ID NO: 33 - DNA, 18 bp, Artificial Sequence
  Description of Artificial Sequence Primer
tcaccaaggccttcttcg

SEQ ID NO: 34 - DNA, 19 bp, Artificial Sequence
  Description of Artificial Sequence Primer
agccgtgggtggcatggtc

SEQ ID NO: 35 - DNA, 18 bp, Artificial Sequence
  Description of Artificial Sequence Primer
aactgataggactcagcg

SEQ ID NO: 36 - DNA, 19 bp, Artificial Sequence
  Description of Artificial Sequence Primer
tccagatcgtcaacaacat

SEQ ID NO: 37 - DNA, 18 bp, Artificial Sequence
  Description of Artificial Sequence Primer
tgagacgtacaccgagac

SEQ ID NO: 38 - DNA, 18 bp, Artificial Sequence
  Description of Artificial Sequence Primer
aggtcacggatgagcaac

SEQ ID NO: 39 - DNA, 30 bp, Artificial Sequence
  Description of Artificial Sequence Primer
attgcggccgctccattcgaggcaaggatc

SEQ ID NO: 40 - DNA, 29 bp, Artificial Sequence
  Description of Artificial Sequence Primer
taagcggccgcagactctggactggagac

SEQ ID NO: 41 - DNA, 27 bp, Artificial Sequence
  Description of Artificial Sequence Primer
taagagctcccattcgaggcaaggatc

SEQ ID NO: 42 - DNA, 27 bp, Artificial Sequence
  Description of Artificial Sequence Primer
tatgatatcgttgccaatgttcaccac

SEQ ID NO: 43 - DNA, 27 bp, Artificial Sequence
  Description of Artificial Sequence Primer
tagactagtatgatgttcaccaaggcc

SEQ ID NO: 44 - DNA, 27 bp, Artificial Sequence
  Description of Artificial Sequence Primer
attgagctcttaggcgccggtggagtg

SEQ ID NO: 45 - DNA, 21 bp, Artificial Sequence
  Description of Artificial Sequence Forward Primer
gcacgtgaaattgttgaaagg

SEQ ID NO: 46 - DNA, 16 bp, Artificial Sequence
  Description of Artificial Sequence Reverse Primer
caggctggccgcattg

SEQ ID NO: 47 - protein, 17 aa, Artificial Sequence
  Description of Artificial Sequence CalA Polypeptide
DQSDVLQFEYTQSGDTI

SEQ ID NO: 48 - protein, 15 aa, Artificial Sequence
  Description of Artificial Sequence CalA Polypeptide
DGGSYSEDWRTNSNG

SEQ ID NO: 49 - protein, 176 aa, Penicillium digitatum
MMFTKTLGLAALFSTIALALPQDMMVRRDGGGVKITNNLAQDVYAWSVSNDVGPMQTITSGGGTYSETWRTNPNGGGISIKLALDPNQQDVLQFEYTEAGDTIFWDMSCINMQAGNNKFTDFGFSVVPSDETSTCPKAICKAGDTACSAAYLKPTDDHATHGCPINTALELSLGQP

SEQ ID NO: 50 - protein, 176 aa, Aspergillus nidulans
MMFTKALVAATLATLTAALPQPTVVRREGGDAGVTIVNNMDSDVYAWSVTDGVSKMHTLSSGGGSYTENFQANPNGGGVSIKLSTHQDQTDVLQFEYTKSGETIFWDMSCIDMDRAASTFTKNGFDVSPSQTSGDCPAVNCHAGDTSCAEAYLQPKDDHATHGCPIDTSFTLTLGA

SEQ ID NO: 51 - protein, 172 aa, Aspergillus terreus
MLFSKLFSLASIAGLSAALPHVERQAASGGVLIVNNLNIPVYAWSVSDTVSPMYTLPAGGGSYTEQWRTNSNGGGISIKLATDPTQADVLQFEYTEAGDTIFWDMSAINLGLDSVFTDYGFFVAPSAADSTCPAVNCQAGDKACSDAYLQPTDDHATHGCPINTFLTLSLGN

SEQ ID NO: 52 - protein, 180 aa, Aspergillus kawachii
MFFKTILTAALATAATALPHSFSNNMVRRNGTSSGSGGVSISNNMSNTTVYAWSVSDRVSNMHTLSAGGGSYSESWQSNDNGGGISIKLSTTESQSDVLQFEYTQSGDTIYWDMSCINLGSDSAFTKYGFSVTPSEEGDNCPSVNCEAGDTECAEAYLQPDDNEATHGCPIDTQFTLTLG

SEQ ID NO: 53 - protein, 180 aa, Aspergillus Niger
MFFKTILTAALATAATALPHSVNNNMVRRNGTSSGSGGVSISNNMSNTTVYAWSVSDRVSNMHTLSAGGGSYSESWQTNDNGGGISIKLSTTESQSDVLQFEYTQDGDTIYWDMSCINLGSDSAFTKYGFSVTPSEEGDNCPSVNCEAGDTACAEAYLQPDDNEATHGCPIDTQFTLTLG

SEQ ID NO: 54 - protein, 172 aa, Aspergillus flavus
MMFTKTITLAALASLAAALPTANPVKRSGGVNIVNNLGETVYAWSVSDRVSNMHTLSANGGNYQESWQTNDNGGGISIKLSNTQEQSNVLQFEYTQSGDTIYWDMSCIDLSGDNVFTKYGFSVTPSSTSDNCPSVNCAAGDTACAEAYLKPNDDHATHGCPIDTSFSVSIGV

SEQ ID NO: 55 - protein, 200 aa, Aspergillus oryzae
MMFTKTITLAALASLAAALPTANPVKRSGGVNIVNNLGETVYAWSVSDRVSNMHTLSANGGNYQESWQTNDNGGGISIKLSNTQEQSNVLQFEYTQSGDTIYWDMSCIDLSGDNVFTKYGFSVTPSSTSDNCPSVNCAAGDTACAEAYLKPNDDHATHGCPIDTSFSSELNPLRLVNMIPTTNMGNGVRYGSGETGKGKN

SEQ ID NO: 56 - protein, 183 aa, Aspergillus ruber
MMFSKIALTMTSFAALTAALPHAIKARNTTSTDSGSGGSGGVSIHNNLEGSVYAWSVSSSAGDMKTLSAGGGSYEESWRENSNGGGVSIKLSTETNQDDVLQFEYTQSGDTIFWDMSCINMGTDSKFVKYGFTVEPSETNDNCPSVNCKAGDSQCAEAYLQPKDDHATHGCPINTQFTVNIGN

SEQ ID NO: 57 - protein, 181 aa, Aspergillus clavatus
MMFSKALVATAFATLTSALPHVMRSGNSTTTESGGGSVQIVNQLDQTVYAWSVADRVSDMHTLSANGGSYTEKWRTNDNGGGISIKLSTKPDQSDVLQFEYTQSDQTIFWDMSCIDLGVQSEFTKYGFSVTPSKSGDKSDCPSVNCHAGDTACAEAYLQPKDDHATHGCPIDTAFTLNIGN

SEQ ID NO: 58 - protein, 177 aa, Aspergillus fumigatus; identical to SEQ ID NO: 1

SEQ ID NO: 59 - protein, 177 aa, Neosartorya fischeri
MMFTKAFLTAAFATLSTALPHVIQRSGNSTASGGGVQIVNNLSQTVYAWSVADSVSDMHTLSADGGSYSEQWRTNSNGGGISIKLSTKPDQSDVLQFEYTQSGDTIFWDMSCIDMGTESEFSKFGFTVEPSQSGGDCPSVNCKAGDTACAEAYLQPKDDHATHGCPIATSFVLNIGN